(19) United States
(12) Patent Application Publication
(10) Pub. No.: US 2026/0317971 A1
(43) Pub. Date: Oct. 15, 2026

(54) Reinforced Nonwoven Material

(71) Applicant: Kimberly-Clark Worldwide, Inc., Neenah, WI (US)

(72) Inventors: Teresa Kubicki, Greensborough, Victoria (AU); Antonio J. Carrillo Ojeda, Appleton, WI (US); Steven J. Roffers, Neenah, WI (US); Dustin J. Smith, Green Bay, WI (US); Folau Piula Penaia, Altona, Victoria (AU); Trudy Chrisley Balchin, Camberwell, Victoria (AU); Justin James Fernandes, Craigieburn, Victoria (AU)

(21) Appl. No.: 19/108,587

(22) PCT Filed: Sep. 21, 2022
(86) PCT No.: PCT/US2022/044262

§ 371 Date: Mar. 4, 2025

(51) Int. Cl. D04H 1/498; D04H 1/492

(52) U.S. Cl. CPC D04H 1/498; D04H 1/492; D10B 2321/021; D10B 2321/022; D10B 2509/026

(57) ABSTRACT

A nonwoven material includes a plurality of fibers forming a non-woven fibrous web. The nonwoven fibrous web includes a perforated zone with a plurality of apertures. The nonwoven fibrous web also includes a plurality of support areas that are configured to limit elongation of the nonwoven fibrous web when the nonwoven fibrous web is under tension.

### BACKGROUND

Nonwoven materials are frequently used within personal care absorbent articles, such as diapers or incontinence briefs. Moreover, an inner lining of such articles face and contact the skin of a wearer. Contact between body exudates, such as semi solid fecal material, captured within the articles and the skin of the wearer can cause discomfort. Moving body exudates through the inner lining and away from the skin of the wearer can reduce or limit such discomfort. Thus, a nonwoven material with features that facilitate movement of body exudates through the nonwoven material would be useful.

Nonwoven materials with apertures can facilitate movement of body exudates through the nonwoven material; however, known methods and mechanisms for forming nonwoven materials with apertures suffer drawbacks. For instance, nonwoven webs can curl under tension, and multilayer webs with apertures tend to curl even more when converted at high-speed. Moreover, the apertures can reduce dimensional and mechanical stability of the web, and apertured webs can thus exhibit larger elongation than that observed in nonapertured webs. Edge curling can cause web fold-overs and flips during high-speed converting, adhesive overspray, and other product issues. Thus, a nonwoven material with features for limiting curling would be useful.

### SUMMARY

In general, the present disclosure is directed to a nonwoven material with features for reducing curling, e.g., while the nonwoven material is under tension. The nonwoven material may include a nonwoven fibrous web with a plurality of support areas. The support areas can limit curling of the nonwoven fibrous web when the nonwoven fibrous web is in tension and thus improve high speed web converting. The support areas may reinforce the nonwoven material by providing increased entanglement and thus bonding between adjacent layers of the nonwoven material. The support areas may thus assist advantageously limit elongation of the nonwoven fibrous web while the nonwoven fibrous web is under tension, which in turn can also limit curling of the nonwoven fibrous web. The nonwoven material with the support areas may be incorporated within an absorbent article, such as a pad, diaper, disposable undergarment, etc.

In one example embodiment, a multilayer nonwoven material includes a laminate nonwoven fibrous web defining a lateral direction and a longitudinal direction. The lateral and longitudinal directions are perpendicular. The laminate nonwoven fibrous web includes a first web and a second web. The laminate nonwoven fibrous web includes a perforated zone with a plurality of apertures that are distributed across a width of the perforated zone along the lateral direction and across a length of the perforated zone along the longitudinal direction. The laminate nonwoven fibrous web includes one or more support areas. Fibers of the first web are entangled within discrete areas in the first web and consolidated with fibers of the second web to a greater extent at the one or more support areas than at the areas of the laminate nonwoven fibrous web adjacent to the one or more support areas. A collective area of the one or more support areas on the laminate nonwoven fibrous web is no less than a half percent and no greater than thirty percent of a total area of the laminate nonwoven fibrous web.

In another example embodiment, a multilayer nonwoven material includes a laminate nonwoven fibrous web defining a lateral direction and a longitudinal direction. The lateral and longitudinal directions are perpendicular. The laminate nonwoven fibrous web includes a first web and a second web. The laminate nonwoven fibrous web includes a perforated zone with a plurality of apertures that are distributed across a width of the perforated zone along the lateral direction and across a length of the perforated zone along the longitudinal direction. The laminate nonwoven fibrous web includes a first surface and a second surface. The first surface is opposite the second surface on the laminate nonwoven fibrous web. The laminate nonwoven fibrous web includes a plurality of nodes extending away from a base plane on the first surface. The laminate nonwoven fibrous web includes one or more support areas. Fibers of the first web are entangled within discrete areas in the first web and consolidated with fibers of the second web to a greater extent at the one or more support areas than at the areas of the laminate nonwoven fibrous web adjacent to the one or more support areas.

In another example embodiment, a multilayer nonwoven material includes a laminate nonwoven fibrous web defining a lateral direction and a longitudinal direction. The lateral and longitudinal directions are perpendicular. The laminate nonwoven fibrous web includes a first web and a second web. The laminate nonwoven fibrous web includes a perforated zone with a plurality of apertures that are distributed across a width of the perforated zone along the lateral direction and across a length of the perforated zone along the longitudinal direction. The laminate nonwoven fibrous web includes a first surface and a second surface. The first surface is opposite the second surface on the laminate nonwoven fibrous web. The laminate nonwoven fibrous web includes a plurality of hollow nodes extending away from a base plane on the first surface. The laminate nonwoven fibrous web includes one or more support areas. Fibers of the first web are entangled within discrete areas in the first web and consolidated with fibers of the second web to a greater extent at the one or more support areas than at the areas of the laminate nonwoven fibrous web adjacent to the one or more support areas. The plurality of support areas are filled by the fibers of the first and second webs between the first and second surfaces of the laminate nonwoven fibrous web.

These and other features, aspects and advantages of the present disclosure will become better understood with reference to the following description and appended claims. The accompanying drawings, which are incorporated in and constitute a part of this specification, illustrate embodiments of the disclosure and, together with the description, serve to explain the principles of the disclosure.

### BRIEF DESCRIPTION OF THE DRAWINGS

A full and enabling disclosure of the present disclosure, including the best mode thereof, directed to one of ordinary skill in the art, is set forth in the specification, which makes reference to the appended figures.

FIG. 1 is a top, plan view of an absorbent article according to an example embodiment of the present disclosure and in a stretched, laid flat, unfastened condition.

FIG. 2 is a top, plan view of a nonwoven material according to an example embodiment of the present disclosure.

FIG. 3 is a partial side, elevation view of support areas of the example nonwoven material of FIG. 2.

FIG. 4 is a schematic side view of an apparatus and process for manufacturing a nonwoven material according to an example embodiment of the present disclosure.

FIG. 4A is a partially exploded view of a formation surface of the example apparatus of FIG. 4.

FIG. 4B is a partial, section view of the formation surface of the example apparatus of FIG. 4.

FIG. 5 is a top, plan view of a nonwoven material according to another example embodiment of the present disclosure.

FIG. 6 is a top, plan view of a nonwoven material according to another example embodiment of the present disclosure.

FIG. 7 is a top, plan view of a nonwoven material according to another example embodiment of the present disclosure.

Repeat use of reference characters in the present specification and drawings is intended to represent the same or analogous features or elements of the present invention.

### DETAILED DESCRIPTION

In an embodiment, the present disclosure is generally directed towards a nonwoven material with support areas, e.g., which may advantageously assist with limiting curling of the nonwoven material while the nonwoven material is under tension. It is to be understood by one of ordinary skill in the art that the present discussion is a description of exemplary embodiments only, and is not intended as limiting the broader aspects of the present disclosure.

When introducing elements of the present disclosure or the preferred embodiment(s) thereof, the articles “a”, “an”, “the” and “said” are intended to mean that there are one or more of the elements. As used herein, the terms “includes” and “including” are intended to be inclusive in a manner similar to the term “comprising.” Similarly, the term “or” is generally intended to be inclusive (i.e., “A or B” is intended to mean “A or B or both”). Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. For example, the approximating language may refer to being within a ten percent (10%) margin.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “fluid entangling”, and “fluid-entangled” generally refers herein to a formation process for further increasing the degree of fiber entanglement within a given fibrous nonwoven web or between fibrous nonwoven webs and other materials so as to make the separation of the individual fibers and/or the layers more difficult as a result of the entanglement. Generally, this is accomplished by supporting the fibrous nonwoven web on some type of forming or carrier surface which has at least some degree of permeability to the impinging pressurized fluid. A pressurized fluid stream (usually multiple streams) is then directed against the surface of the nonwoven web which is opposite the supported surface of the web. The pressurized fluid contacts the fibers and forces portions of the fibers in the direction of the fluid flow thus displacing all or a portion of a plurality of the fibers towards the supported surface of the web. The result is a further entanglement of the fibers in what can be termed the Z-direction of the web (the thickness) relative to its more planar dimension, the X-Y plane. When two or more separate webs or other layers are placed adjacent one another on the forming/carrier surface and subjected to the pressurized fluid, the generally desired result is that some of the fibers of at least one of the webs are forced into the adjacent web or layer thereby causing fiber entanglement between the interfaces of the two surfaces so as to result in the bonding or joining of the webs/layers together due to the increased entanglement of the fibers. The degree of bonding or entanglement will depend on a number of factors including, but not limited to, the types of fibers being used, the fiber lengths, the degree of pre-bonding or entanglement of the web or webs prior to subjection to the fluid entangling process, the type of fluid being used (liquids, such as water, steam or gases, such as air), the pressure of the fluid, the number of fluid streams, the speed of the process, the dwell time of the fluid and the porosity of the web or webs/other layers and the forming/carrier surface. One of the most common fluid entangling processes is referred to as hydroentangling, which is a well-known process to those of ordinary skill in the art of nonwoven webs. Examples of fluid entangling process can be found in U.S. Pat. No. 4,939,016 to Radwanski et al., U.S. Pat. No. 3,485,706 to Evans, and U.S. Pat. Nos. 4,970,104 and 4,959,531 to Radwanski, each of which is incorporated herein in its entirety by reference thereto for all purposes.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article

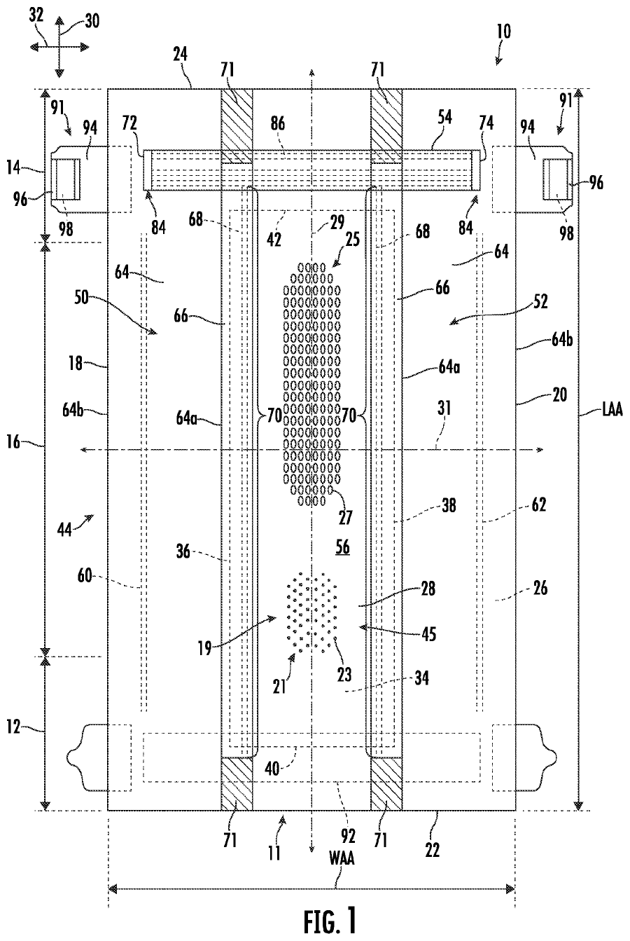

FIG. 1 is a top, plan view of an absorbent article 10 according to an example embodiment of the present disclosure and in a stretched, laid flat, unfastened condition. While absorbent article 10 is shown as a diaper in the example embodiment shown in FIG. 1, it will be understood that absorbent article 10 may be configured as other types of absorbent articles, such as training pants, youth pants, adult incontinence garments, and feminine hygiene articles, and the like, in other example embodiments. While the example embodiments and illustrations described herein may generally apply to absorbent articles manufactured in the product longitudinal direction, which is hereinafter called the machine direction or process direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure.

The absorbent article 10 illustrated in FIG. 1 may include a chassis 11. The absorbent article 10 may also include a front waist region 12, a rear waist region 14, and a crotch region 16 disposed between the front waist region 12 and the rear waist region 14 and interconnecting the front and rear waist regions, 12, 14, respectively. The front waist region 12 may be referred to as the front end region, the rear waist region 14 may be referred to as the rear end region, and the crotch region 16 may be referred to as the intermediate region. With respect to an article manufactured in a cross-direction manufacturing process, for example in a three-piece construction, such an absorbent article may have a chassis including a front waist panel defining the front waist region, a rear waist panel defining the rear waist region, and an absorbent panel defining the crotch region. The absorbent panel may extend between the front waist panel and the rear waist panel. In some example embodiments, the absorbent panel may overlap the front waist panel and the rear waist panel. The absorbent panel may be bonded to the front waist panel and the rear waist panel to define a three-piece construction. However, it is contemplated that an absorbent article may be manufactured in a cross-direction without being a three-piece construction garment.

The absorbent article 10 may have a pair of longitudinal side edges 18, 20, and a pair of opposite waist edges, respectively designated front waist edge 22 and rear waist edge 24. The front waist region 12 may be contiguous with the front waist edge 22 and the rear waist region 14 may be contiguous with the rear waist edge 24. The longitudinal side edges 18, 20 may extend from the front waist edge 22 to the rear waist edge 24. The longitudinal side edges 18, 20 may extend in a direction parallel to the longitudinal direction 30 for their entire length, such as for the absorbent article 10. In other example embodiments, the longitudinal side edges 18, 20 may be curved between the front waist edge 22 and the rear waist edge 24.

The front waist region 12 may include the portion of the absorbent article 10 that, when worn, is positioned at least in part on the front of the wearer while the rear waist region 14 may include the portion of the absorbent article 10 that, when worn, is positioned at least in part on the back of the wearer. The crotch region 16 of the absorbent article 10 may include the portion of the absorbent article 10 that, when worn, is positioned between the legs of the wearer and may partially cover the lower torso of the wearer. The waist edges, 22 and 24, of the absorbent article 10 may be configured to encircle the waist of the wearer and together define a central waist opening for the waist of the wearer. Portions of the longitudinal side edges 18, 20 in the crotch region 16 may generally define leg openings for the legs of the wearer when the absorbent article 10 is worn.

The absorbent article 10 may include an outer cover 26 and a bodyside liner 28. The outer cover 26 and the bodyside liner 28 may form a portion of the chassis 11. In an example embodiment, the bodyside liner 28 may be bonded to the outer cover 26 in a superposed relation by any suitable mechanism such as, but not limited to, adhesives, ultrasonic bonds, thermal bonds, pressure bonds, or other conventional techniques. The outer cover 26 may define a length in a longitudinal direction 30, and a width in the lateral direction 32, which, in the illustrated example embodiment, may coincide with the length LAA and width WAA of the absorbent article 10. As illustrated in FIG. 1, the absorbent article 10 may have a longitudinal axis 29 extending in the longitudinal direction 30 and a lateral axis 31 extending in the lateral direction 32.

The chassis 11 may include an absorbent body 34. The absorbent body 34 may be disposed between the outer cover 26 and the bodyside liner 28. The absorbent body 34 may have longitudinal edges, 36 and 38, which, in an example embodiment, may form portions of the longitudinal side edges, 18 and 20, respectively, of the absorbent article 10. The absorbent body 34 may have a first end edge 40 that is opposite a second end edge 42, respectively, which, in an example embodiment, may form portions of the waist edges, 22 and 24, respectively, of the absorbent article 10. In some example embodiments, the first end edge 40 may be in the front waist region 12. In some example embodiments, the second end edge 42 may be in the rear waist region 14. In an example embodiment, the absorbent body 34 may have a length and width that are the same as or less than the length LAA and width WAA of the absorbent article 10. The bodyside liner 28, the outer cover 26, and the absorbent body 34 may form part of an absorbent assembly 44. In example embodiments of articles according to aspects of the present disclosure, which are manufactured in a cross-direction manufacturing process, the absorbent body 34 may form the absorbent assembly 44. The absorbent assembly 44 may also include a fluid transfer layer (not shown) and/or a fluid acquisition layer (not shown) between the bodyside liner 28 and the absorbent body 34 as is known in the art. The absorbent assembly 44 may also include a spacer layer (not shown) disposed between the absorbent body 34 and the outer cover 26.

The absorbent article 10 may be configured to contain and/or absorb liquid, solid, and semi-solid body exudates discharged from the wearer. In some example embodiments, containment flaps 50, 52 may be configured to provide a barrier to the lateral flow of body exudates. To further enhance containment and/or absorption of body exudates, the absorbent article 10 may suitably include an elasticated waist member 54. In some example embodiments, the elasticated waist member 54 may be disposed in the rear waist region 14 of the absorbent article 10. Although, it is contemplated that the elasticated waist member 54 may be additionally or alternatively disposed in the front waist region 12 of the absorbent article 10.

The elasticated waist member 54 may be disposed on the body facing surface 19 of the chassis 11 to help contain and/or absorb body exudates. In some example embodiments, such as in the absorbent article 10 depicted in FIG. 1, the elasticated waist member 54 may be disposed on the body facing surface 45 of the absorbent assembly 44. In some example embodiments, the elasticated waist member 54 may be disposed at least partially on the body facing surface 56 of the bodyside liner 28.

The absorbent article 10 may further include leg elastic members 60, 62 as are known to those skilled in the art. The leg elastic members 60, 62 may be attached to the outer cover 26 and/or the bodyside liner 28 along the opposite longitudinal side edges, 18 and 20, and positioned in the crotch region 16 of the absorbent article 10. The leg elastic members 60, 62 may be parallel to the longitudinal axis 29 as shown in FIG. 1, or may be curved as is known in the art. The leg elastic members 60, 62 may provide elasticized leg cuffs.

The outer cover 26 and/or portions thereof may be breathable and/or liquid impermeable. The outer cover 26 and/or portions thereof may be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an example embodiment, for example, the outer cover 26 may be constructed of a microporous polymeric film, such as polyethylene or polypropylene.

In an example embodiment, the outer cover 26 may be a single layer of a liquid impermeable material, such as a polymeric film. In an example embodiment, the outer cover 26 may be suitably stretchable, and more suitably elastic, in at least the lateral direction 32 of the absorbent article 10. In an example embodiment, the outer cover 26 may be stretchable, and more suitably elastic, in both the lateral 32 and the longitudinal 30 directions. In an example embodiment, the outer cover 26 may be a multi-layered laminate in which at least one of the layers is liquid impermeable. In some example embodiments, the outer cover 26 may be a two-layer construction, including an outer layer (not shown) and an inner layer (not shown) which may be bonded together such as by a laminate adhesive. Suitable laminate adhesives may be applied continuously or intermittently as beads, a spray, parallel swirls, or the like, but it is to be understood that the inner layer may be bonded to the outer layer by other bonding methods, including, but not limited to, ultrasonic bonds, thermal bonds, pressure bonds, or the like.

The outer layer of the outer cover 26 may be any suitable material and may be one that provides a generally cloth-like texture or appearance to the wearer. An example of such material may be a 100% polypropylene bonded-carded web with a diamond bond pattern available from Sandler A.G., Germany, such as 30 gsm Sawabond 4185® or equivalent. Another example of material suitable for use as an outer layer of an outer cover 26 may be a 20 gsm spunbond polypropylene non-woven web. The outer layer may also be constructed of the same materials from which the bodyside liner 28 may be constructed as described herein.

The liquid impermeable inner layer of the outer cover 26 (or the liquid impermeable outer cover 26 where the outer cover 26 is of a single-layer construction) may be either vapor permeable (i.e., “breathable”) or vapor impermeable. The liquid impermeable inner layer (or the liquid impermeable outer cover 26 where the outer cover 26 is of a single-layer construction) may be manufactured from a thin plastic film. The liquid impermeable inner layer (or the liquid impermeable outer cover 26 where the outer cover 26 is of a single-layer construction) may inhibit liquid body exudates from leaking out of the absorbent article 10 and wetting articles, such as bed sheets and clothing, as well as the wearer and caregiver.

In some example embodiments, where the outer cover 26 is of a single layer construction, it may be embossed and/or matte finished to provide a more cloth-like texture or appearance. The outer cover 26 may permit vapors to escape from the absorbent article 10 while preventing liquids from passing through. A suitable liquid impermeable, vapor permeable material may be composed of a microporous polymer film or a non-woven material which has been coated or otherwise treated to impart a desired level of liquid impermeability.

The absorbent body 34 may be suitably constructed to be generally compressible, conformable, pliable, non-irritating to the wearer's skin and capable of absorbing and retaining liquid body exudates. The absorbent body 34 may be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10. The absorbent body 34 may have a length and width that may be less than or equal to the length LAA and width WAA of the absorbent article 10.

In an example embodiment, the absorbent body 34 may be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an example embodiment, the absorbent body 34 may be a matrix of cellulosic fluff and superabsorbent material. In further example embodiments, the absorbent body 34 may comprise mostly superabsorbent material, or even greater than 80% superabsorbent material, greater than 90% superabsorbent material, or comprise 100% superabsorbent material, by weight of absorbent material of the absorbent body 34. Although, in other example embodiments, the absorbent body 34 may be free of superabsorbent material. In an example embodiment, the absorbent body 34 may be constructed of a single layer of materials, or in the alternative, may be constructed of two or more layers of materials.

Various types of wettable, hydrophilic fibers may be used in the absorbent body 34. Examples of suitable fibers include: natural fibers; cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials may be selected from natural, synthetic, and modified natural polymers and materials. The superabsorbent materials may be inorganic materials, such as silica gels, or organic compounds, such as cross-linked polymers.

If a spacer layer is present, the absorbent body 34 may be disposed on the spacer layer and superposed over the outer cover 26. The spacer layer may be bonded to the outer cover 26, for example, by adhesive. In some example embodiments, a spacer layer may not be present and the absorbent body 34 may directly contact the outer cover 26 and may be directly bonded to the outer cover 26. However, it is to be understood that the absorbent body 34 may be in contact with, and not bonded with, the outer cover 26 and remain within the scope of this disclosure. In an example embodiment, the outer cover 26 may be composed of a single layer and the absorbent body 34 may be in contact with the singer layer of the outer cover 26. In some example embodiments, at least a portion of a layer, such as but not limited to, a fluid transfer layer and/or a spacer layer, may be positioned between the absorbent body 34 and the outer cover 26. The absorbent body 34 may be bonded to the fluid transfer layer and/or the spacer layer.

The bodyside liner 28 of the absorbent article 10 may overlay the absorbent body 34 and the outer cover 26 and may isolate the wearer's skin from liquid waste retained by the absorbent body 34. In various example embodiments, a fluid transfer layer may be positioned between the bodyside liner 28 and the absorbent body 34. In various example embodiments, an acquisition layer (not shown) may be positioned between the bodyside liner 28 and the absorbent body 34 or a fluid transfer layer, if present. In various example embodiments, the bodyside liner 28 may be bonded to the acquisition layer, or to the fluid transfer layer if no acquisition layer is present, via adhesive and/or by a point fusion bonding. The point fusion bonding may be selected from ultrasonic, thermal, pressure bonding, and combinations thereof.

In an example embodiment, the bodyside liner 28 may extend beyond the absorbent body 34 and/or a fluid transfer layer, if present, and/or an acquisition layer, if present, and/or a spacer layer, if present, to overlay a portion of the outer cover 26 and may be bonded thereto by any method deemed suitable, such as, for example, by being bonded thereto by adhesive, to substantially enclose the absorbent body 34 between the outer cover 26 and the bodyside liner 28. It is contemplated that the bodyside liner 28 may be narrower than the outer cover 26. However, in other example embodiments, the bodyside liner 28 and the outer cover 26 may be of the same dimensions in width and length, for example, as may be seen in the example embodiments illustrated in FIG. 1. In other example embodiments, the bodyside liner 28 may be of greater width than the outer cover 26. It is also contemplated that the bodyside liner 28 may not extend beyond the absorbent body 34 and/or may not be secured to the outer cover 26. In some example embodiments, the bodyside liner 28 may wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material. The bodyside liner 28 may be of different shapes, including rectangular, hourglass, or any other shape. The bodyside liner 28 may be suitably compliant, soft feeling, and non-irritating to the wearer's skin and may be the same as or less hydrophilic than the absorbent body 34 to permit body exudates to readily penetrate through to the absorbent body 34 and provide a relatively dry surface to the wearer.

The bodyside liner 28 may be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics may be used for the bodyside liner 28. The bodyside liner 28 may include a woven fabric, a nonwoven fabric, a polymer film, a film-fabric laminate or the like, as well as combinations thereof. Examples of a nonwoven fabric may include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, may include more than one layer of fabrics, films, and/or webs, as well as combinations thereof. For example, the bodyside liner 28 may include a support layer and a projection layer that may be hydroentagled. The projection layer may include hollow projections, such as those disclosed in U.S. Pat. No. 9,474,660 to Kirby, Scott S. C. et al.

For example, the bodyside liner 28 may be composed of a meltblown or spunbond web of polyolefin fibers. Alternatively, the bodyside liner 28 may be a bonded-carded web composed of natural and/or synthetic fibers. The bodyside liner 28 may be composed of a substantially hydrophobic material, and the hydrophobic material may, optionally, be treated with a surfactant or otherwise processed to impart a desired level of wettability and hydrophilicity. The surfactant may be applied by any conventional mechanism, such as spraying, printing, brush coating or the like. The surfactant may be applied to the entire bodyside liner 28, or the surfactant may be selectively applied to particular sections and/or sides of the bodyside liner 28.

In an example embodiment, a bodyside liner 28 may be constructed of a non-woven bicomponent web. The non-woven bicomponent web may be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an example embodiment, a bodyside liner 28 may be a spunbond substrate with a basis weight from about 10 or 12 to about 15 or 20 gsm. In an example embodiment, a bodyside liner 28 may be a 12 gsm spunbond-meltblown-spunbond substrate having 10% meltblown content applied between the two spunbond layers.

Although the outer cover 26 and bodyside liner 28 may include elastomeric materials, it is contemplated that the outer cover 26 and the bodyside liner 28 may be composed of materials which are generally non-elastomeric. In an example embodiment, the bodyside liner 28 may be stretchable, and more suitably elastic. In an example embodiment, the bodyside liner 28 may be suitably stretchable and more suitably elastic in at least the lateral or circumferential direction of the absorbent article 10. In other example aspects, the bodyside liner 28 may be stretchable, and more suitably elastic, in both the lateral and the longitudinal directions 32, 30, respectively.

In an example embodiment, the absorbent article 10 may include a pair of containment flaps 50, 52. The containment flaps 50, 52 may be formed separately from the absorbent chassis 11 and attached to the chassis 11 or may be formed integral to the chassis 11. In some example embodiments, the containment flaps 50, 52 may be secured to the chassis 11 of the absorbent article 10 in a generally parallel, spaced relation with each other laterally inward of the leg openings to provide a barrier against the flow of body exudates. One containment flap 50 may be on a first side of the longitudinal axis 29 and the other containment flap 52 may be on a second side of the longitudinal axis 29. In an example embodiment, the containment flaps 50, 52 may extend generally in a longitudinal direction 30 from the front waist region 12 of the absorbent article 10 through the crotch region 16 to the rear waist region 14 of the absorbent article 10. In some example embodiments, the containment flaps 50, 52 may extend in a direction substantially parallel to the longitudinal axis 29 of the absorbent article 10, however, in other example embodiments, the containment flaps 50, 52 may be curved, as is known in the art.

In example embodiments where the containment flaps 50, 52 are coupled to the chassis 11, the containment flaps 50, 52 may be bonded to the bodyside liner 28 with a barrier adhesive connecting the projections portion 66 to the body facing surface 19 of the chassis 11, or the containment flaps 50, 52 may be bonded to the outer cover 26 with a barrier adhesive in some example embodiments where the bodyside liner 28 does not extend the full lateral width of the outer cover 26. Of course, the containment flaps 50, 52 may be bonded to other components of the chassis 11 and may be bonded with other suitable mechanism other than a barrier adhesive. The containment flaps 50, 52 may be constructed of a fibrous material which may be similar to the material forming the bodyside liner 28. Other conventional materials, such as polymer films, may also be employed.

The containment flaps 50, 52 may each include a base portion 64 and a projection portion 66. The base portion 64 may be bonded to the chassis 11, for example, to the bodyside liner 28 or the outer cover 26 as mentioned above. The base portion 64 may include a proximal end 64a and a distal end 64b. The projection portion 66 may be separated from the base portion 64 at the proximal end 64a of the base portion 64. As used in this context, the projection portion 66 may be separated from the base portion 64 at the proximal end 64a of the base portion 64 in that the proximal end 64a of the base portion 64 defines a transition between the projection portion 66 and the base portion 64. The proximal end 64a of the base portion 64 may be located near the barrier adhesive. In some example embodiments, the distal ends 64b of the base portion 64 may laterally extend to the respective longitudinal side edges 18, 20 of the absorbent article 10. In other example embodiments, the distal ends 64b of the base portion 64 may end laterally inward of the respective longitudinal side edges 18, 20 of the absorbent article 10. The containment flaps 50, 52 may also each include a projection portion 66 that is configured to extend away from the body facing surface 19 of the chassis 11 at least in the crotch region 16 when the absorbent article 10 are in a relaxed configuration. The containment flaps 50, 52 may include a tack-down region 71 in either or both of the front waist region 12 and the rear waist region 14 where the projection portion 66 is coupled to the body facing surface 19 of the chassis 11.

It is contemplated that the containment flaps 50, 52 may be of various configurations and shapes, and may be constructed by various methods. For example, the containment flaps 50, 52 of FIG. 1 depict a longitudinally extending containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10. However, the containment flaps 50, 52 may include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 may be constructed in a “T-shape” configuration, such as described in U.S. Pat. No. 9,259,362 to Robert L. Popp et al. Such a configuration may also include a tack-down region 71 in either or both of the front and rear waist regions 12, 14, respectively. Of course, other configurations of containment flaps 50, 52 may be used in the absorbent article 10 and still remain within the scope of this disclosure.

The containment flaps 50, 52 may include one or more flap elastic members 68, such as the two flap elastic strands depicted in FIG. 1. Suitable elastic materials for the flap elastic members 68 may include sheets, strands or ribbons of natural rubber, synthetic rubber, or thermoplastic elastomeric materials. Of course, while two elastic members 68 are shown in each containment flap 50, 52, it is contemplated that the containment flaps 50, 52 may be configured with one or three or more elastic members 68. Alternatively or additionally, the containment flaps 50, 52 may be composed of a material exhibiting elastic properties itself.

The flap elastic members 68, as illustrated in FIG. 1, may have two strands of elastomeric material extending longitudinally in the projection portion 66 of the containment flaps 50, 52, in generally parallel, spaced relation with each other. The elastic members 68 may be within the containment flaps 50, 52 while in an elastically contractible condition such that contraction of the strands gathers and shortens the projection portions 66 of the containment flaps 50, 52 in the longitudinal direction 30. As a result, the elastic members 68 may bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, when the absorbent article 10 is in a relaxed configuration.

During manufacture of the containment flaps 50, 52 at least a portion of the elastic members 68 may be bonded to the containment flaps 50, 52 while the elastic members 68 are elongated. The percent elongation of the elastic members 68 may be, for example, about 110% to about 350%. The elastic members 68 may be coated with adhesive while elongated to a specified length prior to attaching to the elastic members 68 to the containment flaps 50, 52. In a stretched condition, the length of the elastic members 68 which have adhesive coupled thereto may provide an active flap elastic region 70 in the containment flaps 50, 52, as labeled in FIG. 1, which will gather upon relaxation of the absorbent article 10. The active flap elastic region 70 of containment flaps 50, 52 may be of a longitudinal length that is less than the length LAA of the absorbent article 10. In this exemplary method of bonding the elastic members 68 to the containment flaps 50, 52, the portion of the elastic members 68 not coated with adhesive will retract after the elastic members 68 and the absorbent article 10 are cut in manufacturing to form an individual absorbent article 10. As noted above, the relaxing of the elastic members 68 in the active flap elastic region 70 when the absorbent article 10 is in a relaxed condition may cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28).

Of course, the elastic members 68 may be bonded to the containment flaps 50, 52 in various other ways as known by those of skill in the art to provide an active flap elastic region 70, which is within the scope of this disclosure. Additionally, the active flap elastic regions 70 may be shorter or longer than depicted herein, including extending to the front waist edge 22 and the rear waist edge 24, and still be within the scope of this disclosure.

Leg elastic members 60, 62 may be secured to the outer cover 26, such as by being bonded thereto by laminate adhesive, generally laterally inward of the longitudinal side edges, 18 and 20, of the absorbent article 10. The leg elastic members 60, 62 may form elasticized leg cuffs that further help to contain body exudates. In an example embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28, between the base portion 64 of each containment flap 50, 52 and the outer cover 26, or between the bodyside liner 28 and the outer cover 26. The leg elastic members 60, 62 may be one or more elastic components near each longitudinal side edge 18, 20. For example, the leg elastic members 60, 62 as illustrated herein may each include two elastic strands. A wide variety of elastic materials may be used for the leg elastic members 60, 62. Suitable elastic materials may include sheets, strands or ribbons of natural rubber, synthetic rubber, or thermoplastic elastomeric materials. The elastic materials may be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 may be formed with the containment flaps 50, 52, and then attached to the chassis 11 in some example embodiments. Of course, the leg elastic members 60, 62 may be omitted from the absorbent article 10 without departing from the scope of this disclosure.

In an example embodiment, the absorbent article 10 may have one or more elasticated waist members 54. The elasticated waist member(s) 54 may be disposed in the rear waist region 14 as illustrated in FIG. 1, or in both the rear waist region 14 and the front waist region 12. Although generally described in the present disclosure with reference to a singular elasticated waist member, it should be understood that such description applies equally to each elasticated waist member in example embodiments which contain multiple elasticated waist members 54. As will be discussed in more detail below, the elasticated waist member 54 may help contain and/or absorb body exudates, especially low viscosity fecal matter, and as such, may be preferred to be in the rear waist region 14. An elasticated waist member 54 in the front waist region 12 may help contain and/or absorb body exudates, such as urine, in the front waist region 12. Although not as prevalent as in the rear waist region 14, in some circumstances, fecal material may also spread to the front waist region 12, and thus, an elasticated waist member 54 disposed in the front waist region 12 may help contain and/or absorb body exudates as well.

The elasticated waist member 54 may be comprised of a variety of materials. In a preferred example embodiment, the elasticated waist member 54 may be comprised of a spunbond-meltblown-spunbond (“SMS”) material. However, it is contemplated that the elasticated waist member 54 may be comprised of other materials, such as a spunbond-film-spunbond (“SFS”), a bonded carded web (“BCW”), or any non-woven material. In some example embodiments, the elasticated waist member 54 may be comprised of a laminate of more than one of these exemplary materials, or other materials. In some example embodiments, the elasticated waist member 54 may be comprised of a liquid impermeable material, for example a film material. In some example embodiments, the elasticated waist member 54 may be comprised of a material coated with a hydrophobic coating. The basis weight of the material forming the elasticated waist member 54 may vary, however, in a preferred example embodiment, the basis weight may be between about 8 gsm to about 120 gsm, not including the elastic members 86 in the elasticated waist member 54. More preferably, the basis weight of the material comprising the elasticated waist member 54 may be between about 10 gsm to about 40 gsm, and even more preferably, between about 15 gsm to about 25 gsm.

The elasticated waist member 54 may include a first longitudinal side edge 72, a second longitudinal side edge 74, a waist member first end edge, and a waist member second end edge joining the first longitudinal edge 72 and the second longitudinal edge 74. The first longitudinal side edge 72 may be opposite from the second longitudinal side edge 74. The distance between the first longitudinal side edge 72 and the second longitudinal side edge 74 may define a width of the elasticated waist member 54 in the lateral direction 32. Although not depicted, in some example embodiments, the first longitudinal side edge 72 may substantially align with the first longitudinal side edge 18 of the absorbent article 10. Similarly, in some example embodiments, the second longitudinal side edge 74 may align with the second longitudinal side edge 20 of the absorbent article 10. As illustrated in FIG. 1, the elasticated waist member 54 may be configured such that the first longitudinal side edge 72 may be disposed laterally outward of the proximal end 64a of the base portion 64 of the containment flap 50. Similarly, the elasticated waist member 54 may be configured such that the second longitudinal side edge 74 may be disposed laterally outward of the proximal end 64a of the base portion 64 of the containment flap 52.

In preferred example embodiments, the elasticated waist member 54 may include at least one elastic member 86. In some example embodiments, the elasticated waist member 54 may include multiple elastic members 86, such as five elastic members 86. Of course, it is contemplated that the elasticated waist member 54 may include other numbers of elastic members 86, such as three, four, six, eight, or ten elastic members, and in some example embodiments, no elastic members 86. The elastic member 86 may span substantially from the first longitudinal side edge 72 to the second longitudinal side edge 74 of the elasticated waist member 54. In some example embodiments, the elastic members 86 may be spaced evenly in the longitudinal direction 30. At least one of the elastic members 86 may be disposed located near the waist member second end edge of the elasticated waist member 54.

A wide variety of elastic materials may be used for the elastic member(s) 86 in the elasticated waist member 54. Suitable elastic materials may include sheets, strands or ribbons of natural rubber, synthetic rubber, thermoplastic elastomeric materials, or elastic foams. The elastic materials may be stretched and secured to a substrate forming the elasticated waist member 54, secured to a gathered substrate, or secured to a substrate and then elasticized or shrunk, for example, with the application of heat, such that the elastic retractive forces are imparted to the substrate forming the elasticated waist member 54.

In some example embodiments, the elasticated waist member 54 may be disposed on the body facing surface 45 of the absorbent assembly 44. In some example embodiments, such as in example embodiments illustrated in FIG. 1, the elasticated waist member 54 may be disposed on the body facing surface 56 of the bodyside liner 28.

In various example embodiments, the elasticated waist member 54 may also include a proximal portion (not shown) and a distal portion (not shown). The proximal portion may be coupled to the body facing surface 19 of chassis 11 (e.g., the body facing surface 45 of the absorbent assembly 44 or the body facing surface 56 of the bodyside liner 28) whereas the distal portion or at least a portion of the distal portion of the elasticated waist member 54 may be free to move with respect to the chassis 11 and the absorbent assembly 44 when the absorbent article 10 is in the relaxed configuration. A first fold (not shown) may separate the proximal portion from the distal portion in the various example embodiments of the elasticated waist member 54 discussed herein. As used in this context, the first fold separates the proximal portion from the distal portion in that the first fold defines a transition between the proximal portion and the distal portion in the elasticated waist member material and the elasticated waist member 54 as a whole. In alternate example embodiments (not shown), the proximal portion and the distal portion may be made from separate materials which are attached to each other such as, for example, in the area of the first fold or in lieu of the first fold. The physical form of the attachment may be, for example, by way of a butt seam or a lap seam.

The proximal portion of such an elasticated waist member 54 may be coupled to the body facing surface 19 of the chassis 11 with an adhesive, and in some example embodiments, the proximal portion may be coupled to the body facing surface 45 of the absorbent assembly 44. In some example embodiments, the proximal portion of the elasticated waist member 54 may be coupled to the body facing surface 56 of the bodyside liner 28. However, in some example embodiments, the proximal portion of the elasticated waist member 54 may be coupled to the body facing surface 58 of the rear waist panel 15. The proximal portion may be coupled to the body facing surface 45 of the absorbent assembly 44 with adhesive along the entire length of the proximal portion in the longitudinal direction 30. However, it can be contemplated that only a portion of the proximal portion in the longitudinal direction 30 may be coupled to the body facing surface 45 of the absorbent assembly 44. Of course, it is contemplated that the proximal portion of the elasticated waist member 54 may be coupled to the body facing surface 19 of the chassis 11 or the body facing surface 45 of the absorbent assembly 44 by means other than an adhesive, such as by pressure bonding, ultrasonic bonding, thermal bonding, and combinations thereof. In preferred example embodiments, the proximal portion is coupled to the body facing surface 19 of the chassis 11 in the lateral direction 32 in a constant fashion along the lateral axis 31, as opposed to an intermittent fashion, such that a barrier to body exudates is formed between the proximal portion and the body facing surface 19 of the chassis 11.

The proximal portion of the elasticated waist member 54 may include a longitudinal length measured in the longitudinal direction 30 along the longitudinal axis 29 that is shorter than a longitudinal length of the distal portion of the elasticated waist member 54 (not shown). However, in some example embodiments, the longitudinal length of the proximal portion may be substantially equal to or larger than the longitudinal length of the distal portion of the elasticated waist member 54. It can be appreciated that the relative longitudinal lengths of the proximal portion and the distal portion may be varied between example embodiments of the elasticated waist member 54 without departing from the scope of this disclosure.

In such example embodiments of an elasticated waist member 54, because the distal portion of the elasticated waist member 54 can freely move with respect to the absorbent assembly 44 when the absorbent article 10 is in the relaxed configuration, the distal portion may assist with providing a containment pocket when the absorbent article 10 is in the relaxed configuration when being worn by the wearer. The containment pocket may assist with providing a barrier to contain and/or absorb body exudates. The first longitudinal side edge 72 may be disposed laterally outward of the proximal end 64a of the base portion 64 of the containment flap 50, and thus, the pocket may extend laterally outward of the proximal end 64a of the containment flap 50. Similarly, the second longitudinal side edge 74 may be disposed laterally outward of the proximal end 64a of the base portion 64 of the containment flap 52 and the pocket may extend laterally outward of the proximal end 64a of the containment flap 52. Such a configuration provides elasticated waist member 54 with a wide containment pocket to contain and/or absorb body exudates. To help prevent lateral flow of body exudates that are contained by the containment pocket of the elasticated waist member 54, the distal portion of the elasticated waist member 54 may be bonded to the proximal portion of the elasticated waist member 54 and/or the body facing surface 19 of the chassis 11 near the first and second longitudinal side edges 72, 74, respectively. For example, FIG. 1 depicts tack-down regions 84 where the distal portion of the elasticated waist member 54 may be bonded to the proximal portion of the elasticated waist member 54 and/or the body facing surface 19 of the chassis 11 near the first and second longitudinal side edges 72, 74, respectively.

As depicted in FIG. 1, in some example embodiments the elasticated waist member 54 may be disposed on the body facing surface 19 of the chassis 11 such that a gap is provided between the second end edge 42 of the absorbent body 34 and the waist member second end edge of the distal portion of the elasticated waist member 54. By providing a gap, the containment may have a greater void volume for body exudates. Additionally, it is believed that gap can help body exudates enter the containment pocket of the elasticated waist member 54.

The elasticated waist member 54 may be disposed to be coupled to the chassis 11 by being placed either over the containment flaps 50, 52 or under the containment flaps 50, 52. More specifically, as shown in FIG. 1, the elasticated waist member 54 may be disposed on the body facing surface 19 of the chassis 11 such that the proximal portion of the elasticated waist member 54 is disposed over the base portion 64 of the first and the second containment flaps 50, 52, respectively. Alternatively, the elasticated waist member 54 may be disposed on the body facing surface 19 of the chassis 11 such that the proximal portion of the elasticated waist member 54 is disposed under the base portion 64 of the first and the second containment flaps 50, 52, respectively. Both configurations can provide advantages to the functioning of the elasticated waist member 54 to contain and/or absorb body exudates.

Example embodiments where the proximal portion of the elasticated waist member 54 is disposed over the base portion 64 of the containment flaps 50, 52 can provide the advantage that the containment flaps 50, 52 assist the distal portion of the elasticated waist member 54 extend away from the body facing surface 45 of the absorbent assembly 44 when the absorbent article 10 is applied to the wearer. This is especially relevant where the proximal portion of the elasticated waist member 54 has a shorter longitudinal length than the distal portion of the elasticated waist member 54. For example, when the proximal portion is shorter than the distal portion, the flap elastics 68 in the projection portion 66 of the containment flaps 50, 52 can provide an opening force on the distal portion of the elasticated waist member 54 when the absorbent article 10 is in the relaxed configuration and applied to the wearer, thus helping the distal portion extend away from the body facing surface 45 of the absorbent assembly 44 and opening the containment pocket. In some example embodiments, the containment pocket may be additionally or alternatively opened by configuring the containment flaps 50, 52 to have an active flap elastic region 70 that longitudinally overlaps with the distal portion of the elasticated waist member 54 when the absorbent article 10 is in the stretched, laid flat configuration, such as illustrated in FIG. 1. Additionally or alternatively, the containment pocket of the elasticated waist member 54 may be opened by configuring the containment flaps 50, 52 to have a tack-down region 71 that does not extend to a distal edge of the distal portion of the elasticated waist member 54, such as illustrated in FIG. 1. However, such a configuration of the tack-down region 71 is not required, and in some example embodiments, the tack-down region 71 may extend from the rear waist edge 24 past the distal edge of the distal portion of the elasticated waist member 54.

Example embodiments where the proximal portion of the elasticated waist member 54 is disposed under the base portion 64 of the containment flaps 50, 52 can provide the advantage of having the containment pocket formed by the elasticated waist member 54 be free from the projection portion 66 of the containment flaps 50, 52. Both the base portion 64 and the projection portion 66 of each containment flap 50, 52 may be coupled to the body facing surface 55 of the elasticated waist member 54. As a result, body exudates may more freely spread through the full width of the containment pocket created by the elasticated waist member 54. Additionally, the coupling of the base portion 64 of the containment flaps 50, 52 to the outer cover 26 (or in some example embodiments to the bodyside liner 28) may create a longitudinal barrier to the flow of body exudates out of the containment pocket for exudates that spread laterally beyond the location of a barrier adhesive connecting the projection portion 66 of the flaps 50, 52 to the body facing surface 19 of the chassis 11. In some example embodiments, the tack-down region 71 of the projection portion 66 of each of the containment flaps 50, 52 may longitudinally overlap with the distal portion of the elasticated waist member 54. In some example embodiments, the tack-down region 71 of projection portion 66 of each of the containment flaps 50, 52 may extend to the distal edge of the elasticated waist member 54 to further assist in containing exudates within the containment pocket created by the elasticated waist member 54. For instance, the containment pocket and other components of absorbent article 10 may be formed in the same or similar manner to that described in U.S. Pat. No. 10,159,610, which is incorporated by reference herein in its entirety for all purposes.

In example embodiments, the absorbent article 10 may include a fastening system. The fastening system may include one or more back fasteners 91 and one or more front fasteners 92. The example embodiments being shown in FIG. 1 depict example embodiments with one front fastener 92. Portions of the fastening system may be included in the front waist region 12, rear waist region 14, or both.

The fastening system may be configured to secure the absorbent article 10 about the waist of the wearer in a fastened condition and help maintain the absorbent article 10 in place during use. In an example embodiment, the back fasteners 91 may include one or more materials bonded together to form a composite ear as is known in the art. For example, the composite fastener may be composed of a stretch component 94, a nonwoven carrier or hook base 96, and a fastening component 98, as labeled in FIG. 1. In some example embodiments, the elasticated waist member 54 may laterally extend to the back fasteners 91, and/or to each of the longitudinal side edges 18, 20 of the absorbent article 10. In some example embodiments, the elasticated waist member 54 may be coupled to the stretch component 94 of the back fasteners 91, either directly or indirectly.

According to some example embodiments of the present disclosure, bodyside liner 28 of the article 10 may further include zones with different morphological features. In various example embodiments, the morphological features may include one or more of: discrete perforated zones; discrete depression and/or protrusion zones, e.g., with depressions and/or hollow protrusions (such as those formed by embossing or other such web-modification processes); discrete apertures (whether integrally formed during web formation or formed through post web-formation processes); and/or discrete filled protrusions extending above, or below depending on orientation, a generally planar surface of the web.

As a particular example, bodyside liner 28 may include a first feature zone 21 with one or more first features 23 and a second feature zone 25 with one or more second features 27. In the example embodiment shown in FIG. 1, the features 23, 27 may be apertures that extend through bodyside liner 28. The apertures 23, 27 may assist with transferring body exudates through the bodyside liner 28 into interior portions of the article 10 where the exudates are stored and disposed away from a wearer's skin. However, while described in greater detail below in the context of apertures, it will be understood that each of first and second feature zones 21, 25 may include one or more alternative structural features in other example embodiments. For instance, each of first and second feature zones 21, 25 may include one or more of embossments, projections, depressions, perforations, protrusions, recesses, apertures, and the like. Thus, each of first and second feature zones 21, 25 may include discrete structural features 23, 27 on bodyside liner 28, and the structural features 23 in first feature zone 21 may be spaced apart from the structural features 27 in second feature zone 25 on bodyside liner 28. In certain example embodiments, the structural features 23 in first feature zone 21 may be sized and/or formed differently than the structural features 27 in second feature zone 25. Thus, e.g., the structural features 23 in first feature zone 21 may provide different performance characteristics than the structural features 27 in second feature zone 25. Moreover, each of first and second feature zones 21, 25 may be separately arranged, sized, shaped, and/or configured to provide a respective performance characteristic therein. As another example, the features 27 of second feature zone 25 may provide a different visual appearance than the features 23 in first feature zone 21, such as printed dots or wavy pattern, e.g., to assist a user with distinguishing between first and second zones 21, 25.

Portions of the bodyside liner 28 including the apertures 27 may be particularly suited to transferring and trapping low-viscosity fecal matter away from a wearer's skin. Such an effect may help to maintain comfort and skin health of a wearer by preventing prolonged contact between the fecal matter and the skin of a wearer of article 10. Portions of the bodyside liner 28 including the apertures 27, or other relatively large apertures, may be less desirable for management of urine exudate. For example, where apertures 27 are large enough or plentiful enough to provide a relatively large open area of the liner 28, such apertures 27 provide an avenue for urine to seep back to the body facing surface 19 of the article 10 and thus in contact with a wearer's skin-potentially causing discomfort and/or skin health issues.

In at least some example embodiments, the second feature zone 25 may be disposed in a localized region of the article 10, as shown in FIG. 1. For example, the second feature zone 25 may have a longitudinal extent that is less than the longitudinal extent LAA of the article 10. More specifically, the second feature zone 25 may have a longitudinal extent that less than seventy percent (70%), such as less than sixty percent (60%), such as less than fifty percent (50%), such as about less than forty-seven percent (47%), of the longitudinal extent LAA of the article 10. Additionally, the second feature zone 25 may have a lateral extent that is less than the lateral extent WAA of the article 10. In some of these example embodiments, the second feature zone 25 may be disposed wholly between proximal ends 64a of base portions 64 of the containment flaps 50, 52. In some example embodiments, the second feature zone 25 may be located wholly within the crotch region 16. In other example embodiments, the second feature zone 25 may be located wholly within the rear waist region 14. In still further example embodiments, the second feature zone 25 may span both a portion of the crotch region 16 and the rear waist region 14. In still further example embodiments, the second feature zone 25 may be located between no less than forty percent (40%) and no greater than eighty percent (80%) of the longitudinal extent LAA of the article 10 from the front waist edge 22. In certain example embodiments, the second feature zone 25 may also extend across the lateral axis 31, e.g., such that the second feature zone 25 at least partially overlaps the lateral axis 31 within the crotch region 16. The crotch region 16 and the rear waist region 14 are the locations within the article 10 where fecal matter is typically received.

Portions of the bodyside liner 28 forming the first feature zone 21 may be particularly suited to handling fluids, such as urine,—for example, transferring and trapping liquid away from the skin of a wearer of the article 10. Such an effect may help to maintain comfort and skin health of the wearer by preventing prolonged contact between the urine and the skin of the wearer of the article 10. As noted above, the first feature zone 21 may accomplish such function through the use of individual structural features or any combination of structural features. In at least some example embodiments, the structural features 23 of the first feature zone 21 include apertures. Where the features 23 include apertures, such apertures may be relatively small apertures for management of urine exudate.

Accordingly, in at least some example embodiments, the first feature zone 21 may be disposed in a localized region of the article 10, as shown in FIG. 1. For example, the first feature zone 21 may have a longitudinal extent that is less than the longitudinal extent LAA of the article 10. More specifically, the first feature zone 21 may have a longitudinal extent that is less half, or less than a third, or less than a quarter, of the longitudinal extent LAA of the article 10. Additionally, the first feature zone 21 may have a lateral extent that is less than the lateral extent WAA of the article 10. In some of these example embodiments, the first feature zone 21 may be disposed wholly between proximal ends 64a of base portions 64 of the containment flaps 50, 52. In some example embodiments, the first feature zone 21 may be located wholly within the crotch region 16. The crotch region 16 is the location within the article 10 where urine is typically received.

### Three-Dimensional Web With Support Areas

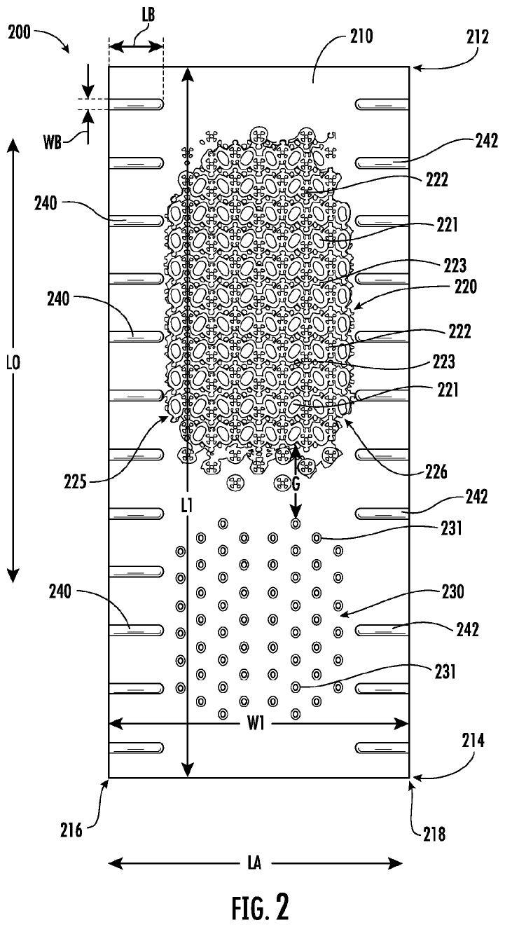

FIG. 2 is a top, plan view of a nonwoven material 200 according to an example embodiment of the present disclosure. In FIG. 2, nonwoven material 200 is shown in a flat or planar configuration. Nonwoven material 200 may be used in absorbent article 10, e.g., as bodyside liner 28, Thus, nonwoven material 200 is described in greater detail below in the context of absorbent article 10. However, it will be understood that nonwoven material 200 may be used in any other article or garment in alternative example embodiments. For instance, nonwoven material 200 may be used in diapers, diaper pants, training pants, youth pants, swim pants, feminine hygiene products, including, but not limited to, menstrual pads or pants, incontinence products, medical garments, surgical pads and bandages, other personal care or health care garments, e.g., as an inner liner facing a wearer of such products, and wipes. As discussed in greater detail below, nonwoven material 200 may advantageously include features for limiting or reducing curling of nonwoven material 200 while under tension, e.g., notwithstanding the presence of other features, such as apertures, nodes, etc., on nonwoven material 200.

As shown in FIG. 2, nonwoven material 200 includes a nonwoven fibrous web 210, e.g., formed with a plurality of fibers. Fibrous web 210 may define a lateral direction LA and a longitudinal direction LO. The lateral and longitudinal directions LA, LO may be perpendicular to each other. In certain example embodiments, longitudinal direction LO may correspond to the longitudinal direction 30, and lateral direction LA may correspond to the lateral direction 32. Fibrous web 210 may extend between a front or first end portion 212 and a rear or second end portion 214, e.g., along the longitudinal direction LO. Thus, first and second end portions 212, 214 may be spaced apart along the longitudinal direction LO. First end portion 212 of fibrous web 210 may be positioned at or adjacent rear waist region 14 of absorbent article 10, and second end portion 214 of fibrous web 210 may be positioned at or adjacent front waist region 12 of absorbent article 10. Fibrous web 210 may also extend between a first side portion 216 and a second side portion 218, e.g., along the lateral direction LA. Thus, first and second side portions 216, 218 may be spaced apart along the lateral direction LA.

A length L1 of fibrous web 210 may be defined between first and second end portions 212, 214 of fibrous web 210, and a width W1 of fibrous web 210 may be defined between first and second side portions 216, 218 of fibrous web 210. In certain example embodiments, the length L1 of fibrous web 210 may be greater than the width W1 of fibrous web 210. For example, the length L1 of fibrous web 210 may be no less than two times (2×) greater than the width W1 of fibrous web 210, such as no less than three times (3×) greater than the width W1 of fibrous web 210. Thus, fibrous web 210 may be elongated along longitudinal direction LO between the first and second end portions 212, 214.

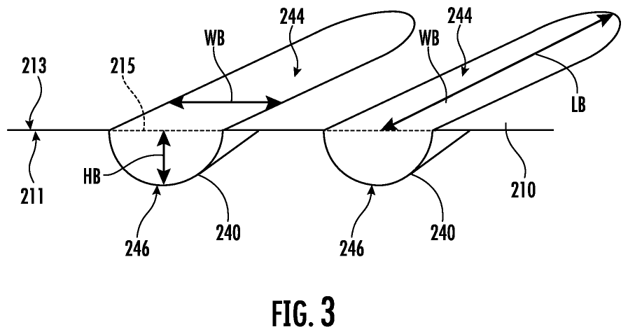

Nonwoven fibrous web 210 may include at least one feature zone. For instance, as shown in FIG. 3, nonwoven fibrous web 210 may include a first feature zone 220 and a second feature zone 230. First feature zone 220 may be spaced apart from second feature zone 230, e.g., along the longitudinal direction LO. For example, first and second feature zones 220, 230 may be spaced apart by a gap G along the longitudinal direction LO. In certain example embodiments, the gap G may be no less than six millimeters (6 mm) and no greater than one hundred and thirty millimeters (130 mm), such as no less than twelve millimeters (12 mm) and no greater than one hundred millimeters (100 mm), such as no less than twenty-five millimeters (25 mm) and no greater than seventy-five millimeters (75 mm). As shown in FIG. 2, nonwoven fibrous web 210 may be unperforated between first and second feature zones 220, 230, e.g., along the longitudinal direction LO. Thus, e.g., the nonwoven fibrous web 210 may not be processed to include apertures between first and second feature zones 220, 230 along the longitudinal direction LO. In certain example embodiments, a centroid of first feature zone 220 may also be arranged colinear with a centroid of second feature zone 230, e.g., on longitudinal axis 29. Moreover, first and second feature zones 220, 230 may be positioned at a middle portion of nonwoven fibrous web 210, e.g., along the lateral direction LA, between first and second side portions 216, 218 of fibrous web 210.

First feature zone 220 may include a plurality of apertures 221 and a plurality of nodes 222, and second feature zone 230 may also include a plurality of apertures 231. Apertures 221 of first feature zone 220 may be larger than apertures 231 of second feature zone 230. For example, an area of each of apertures 221 of first feature zone 220 may be no less than about five millimeters squared (5 mm²) and no greater than about twenty-eight millimeters squared (28 mm²), such as no less than about eight millimeters squared (8 mm²) and no greater than about twenty millimeters squared (20 mm²), such as no less than about nine millimeters squared (9 mm²) and no greater than about twelve millimeters squared (12 mm²). In contrast, an area of each of apertures 231 of second feature zone 230 may be no less than about a quarter millimeter squared (0.25 mm²) and no greater than about five millimeters squared (5 mm²), such as no less than about one millimeter squared (1 mm²) and no greater than about four millimeters squared (4 mm²), such as about two and a half millimeters squared (2.5 mm²). Such sizing differential between apertures 221 of first feature zone 220 and apertures 231 of second feature zone 230 may advantageously facilitate movement of body exudates through nonwoven fibrous web 210. Moreover, the larger apertures 221 of first feature zone 220 may be sized for transferring and trapping low-viscosity fecal matter. Conversely, the smaller apertures 231 of second feature zone 230 may be sized for transferring and trapping urine exudate. In certain example embodiments, apertures 221 may be distributed throughout first feature zone 220, and apertures 231 may be distributed throughout second feature zone 230. For instance, apertures 221 may be uniformly distributed throughout first feature zone 220, and apertures 231 may be uniformly distributed throughout second feature zone 230.

As noted above, nonwoven material 200 may include nodes 222 at first feature zone 220. It will be understood that nonwoven material 200 may include nodes at other locations on nonwoven material 200 in alternative example embodiments. Moreover, it will be understood that, in certain example embodiments, first feature zone 220 may include nodes 222 without apertures 221 or vice versa. As shown in FIG. 2, nonwoven fibrous web 210 may also include a plurality of connecting ligaments 223 at first feature zone 220. The nodes 222 may extend away from a base plane 215 on a first surface 211 (FIG. 3) of the nonwoven material 200. The base plane 215 may be defined as the generally planar region of the first surface 211 of the nonwoven material 200 other than the portion of the nonwoven material 200 forming the nodes 222. In other words, for the example embodiment depicted in FIGS. 2 and 3, the base plane 215 may be formed by the first surface 211 of the nonwoven material 200 that provides the connecting ligaments 223. The nonwoven material 200 may also include a second surface 213. The first surface 211 may be opposite from the second surface 213 on nonwoven fibrous web 210, as depicted in FIG. 3.

The nodes 222 may be configured in a variety of shapes and sizes. In some example embodiments, the nodes 222 may be generally cylindrical in shape. In certain example embodiments, the nodes 222 may be configured to not include any openings or apertures. In certain example embodiments, the nodes 222 may have a height (as measured in a direction perpendicular to the base plane 215, e.g., from base plane 215 to a distal end portion of node 222) of between about a half millimeter (0.5 mm) to about ten millimeters (10 mm), such as from about one millimeter (1 mm) to about three millimeters (3 mm), such as from about one and one-tenth millimeter (1.1 mm) to about one and five-tenths millimeters (1.5 mm), such as about one and three-tenths millimeters (1.3 mm). In certain example embodiments, a majority of the nodes 222 may each have an area (as measured by the area of the node 222 at the base plane 215) from about five millimeters squared (5 mm²) to about thirty-five millimeters squared (35 mm²), such as from about ten millimeters squared (10 mm²) to about twenty millimeters squared (20 mm²). The nodes 222 may be configured in the first feature zone 220 such that the nodes 222 provide a node density of about one node per square centimeter (1.0 nodes/cm²) to about three nodes per square centimeter (3.0 nodes/cm²).

As depicted in FIG. 2, the connecting ligaments 223 may interconnect the nodes 222. Moreover, each connecting ligament 223 may be referred to as extending between only two (2) adjacent nodes 222. In other words, an individual, discrete connecting ligament 223 may not interconnect three (3) or more nodes 222. In this manner, such node and ligament structure differs from an “islands-in-the-sea” configuration where the nodes are dispersed between continuously extending and interconnected ‘land areas’. In example embodiments, a majority of nodes 222 may include at least three (3) connecting ligaments 223 connecting to adjacent nodes 222. In certain example embodiments, a majority of nodes 222 may include ten (10) or less connecting ligaments 223 connecting to adjacent nodes 222. In some example embodiments, the nonwoven material 200 may be configured such that a majority of nodes 222 may include three (3) to eight (8) connecting ligaments 223 connecting to adjacent nodes 222. For example, in the example embodiment in FIG. 2, a majority of the nodes 222 include four connecting ligaments 223 that connect to adjacent nodes 222.

The apertures 221 may be areas of the nonwoven fibrous web 210 that have a lower density of fibers of the nonwoven fibrous web 210 in comparison to nodes 222 and connecting ligaments 223. In some example embodiments, the apertures 221 may be substantially devoid of fibers. As used herein, the apertures 221 are to be distinguished from the normal interstitial fiber-to-fiber spacing commonly found in fibrous nonwoven materials. For example, the apertures 221 may include a lower density of fibers than adjacent nodes 222 and connecting ligaments 223. The apertures 221 may be formed between the connecting ligaments 223 and the nodes 222. Individual apertures 221 may be disposed between adjacent nodes 222. In certain example embodiments, each aperture 221 may be defined between at least three (3) connecting ligaments 223 and at least three (3) nodes 222. In some example embodiments, individual, discrete apertures 221 may be defined between at least four (4) connecting ligaments 223 and at least four (4) nodes 222.

As shown in FIGS. 2 and 3, nonwoven material 200 may include a plurality of support areas 240. Support areas 240 may advantageously assist with reducing curling, e.g., while the nonwoven material 200 is under tension. For example, nonwoven material 200 may be a multilayer nonwoven material 200 with two materials, e.g., two webs laminated together and having different elongation properties. When under tension, the two materials of the multilayer nonwoven material 200 may act differently-due to differing mechanical properties-resulting in edge curling of the multilayer nonwoven material 200 (e.g., at first and second side portions 216, 218), which can lead to web fold-overs and flips during high-speed converting, adhesive overspray, and other product issues. Apertures 221 within first perforated zone 220 may also reduce the dimensional and mechanical stability of the nonwoven material 200 relative to the other portions of the nonwoven material 200 without apertures and/or with smaller apertures. This reduced dimensional and mechanical stability of a nonwoven material with apertures like apertures 221 within first perforated zone 220 can also lead to curling of the nonwoven material under tension, e.g., due to elongation, which can lead to web fold-overs and flips during high-speed converting, adhesive overspray, and other product issues. When the nonwoven material 200 is a multilayer nonwoven material 200, the edge curling may be particularly pronounced due to the combination of the differing mechanical properties of the two materials in the multilayer nonwoven material 200 and the reduced dimensional and mechanical stability associated with the apertures 221 within first perforated zone 220. The support areas 240 on the nonwoven material 200 may advantageously reduce or limit curling of the nonwoven material 200, e.g., due to the differing mechanical properties at discrete areas within the multilayer nonwoven material 200 which may consequently reduce dimensional and mechanical stability associated with the apertures 221 within first perforated zone 220.

Support areas 240 may correspond to areas of the multilayer nonwoven material 200 with more or greater bonding within a first web/layer and between the two materials in the multilayer nonwoven material 200 relative to other areas of the multilayer nonwoven material 200, such as unperforated regions of the multilayer nonwoven material 200 (e.g., immediately) adjacent the support areas 240. For instance, the two materials in the multilayer nonwoven material 200 at support areas 240 may have more or greater entanglement between the fibers within the discrete support areas 240 than other areas of the multilayer nonwoven material 200, such as unperforated regions of the multilayer nonwoven material 200 (e.g., immediately) adjacent the support areas 240. Thus, e.g., a greater portion of the fibers of a first web of the multilayer nonwoven material 200 at the support areas 240 may be entangled together and a greater portion of fibers of the first web and a second web may be consolidated together at an interface of the first web and the second web at the support areas 240 than at nodes 222 and/or at unperforated portions of the multilayer nonwoven material 200. By increasing the bonding of fibers of the first web and between the two webs in the multilayer nonwoven material 200 at support areas 240, the differing elongation properties of the two materials-due to differing mechanical properties-may advantageously be reduced such that the curling of the multilayer nonwoven material 200 under tension is also reduced. Moreover, the greater degree of bonding at discrete areas with the multilayer nonwoven material 200 at support areas 240, the more that the multilayer nonwoven material 200 may perform like a nonwoven material 200 with one material or web with regards to edge curling by reducing the differential between the elongation properties of the two materials.

The support areas 240 on nonwoven material 200 may correspond to regions of the nonwoven material 200 having a different basis weight, different density, different caliper (e.g., material height), and/or different a level of fiber-to-fiber bonding than adjacent areas of nonwoven material 200. Thus, e.g., a basis weight of nonwoven material 200 at support areas 240 may be greater than (e.g., no less than ten percent (10%) greater than and/or no greater than ninety percent (90%) greater than) the basis weight of nonwoven material 200 at areas of nonwoven material 200 (e.g., immediately) adjacent support areas 240. As another example, the caliper of nonwoven material 200 at support areas 240 may be greater than (e.g., no less than ten percent (10%) greater than and/or no greater than ninety percent (90%) greater than) the caliper of nonwoven material 200 at areas of nonwoven material 200 (e.g., immediately) adjacent support areas 240. As yet another example, the density of nonwoven material 200 at support areas 240 may be greater than (e.g., no less than ten percent (10%) greater than and/or no greater than ninety percent (90%) greater than) the density of nonwoven material 200 at areas of nonwoven material 200 (e.g., immediately) adjacent support areas 240. Support areas 240 may thus have different material or mechanical properties than adjacent areas of nonwoven material 200, e.g., in order to provide increased localized strength and/or rigidity relative to the adjacent areas of nonwoven material 200.

As may be seen from the above, due to the increased bonding at discrete areas and between the two materials, support areas 240 may advantageously reduce curling of the multilayer nonwoven material 200. Support areas 240 may also provide increased strength to the nonwoven material 200. For example, the support areas 240 can act to reinforce the material 200 in a manner similar to an I-beam, in order increase a rigidity of the material 200. This increased resistance to elongation under tension helps to reduce necking which in turns contributes to edge fold-over. It is further believed that example embodiments of the present disclosure that include support areas 240 disposed adjacent the side regions of the material 200 provide increased strength concentrated particularly in those edge regions. Accordingly, in addition to helping with overall strength under tension, it is believed that such support areas 240 can provide increased resistance to bending of the material 200 in the side regions and thereby also contributing to a reduction in fold-overs when under tension and during converting.

It will be understood that support areas 240 may have various sizes, locations, and orientation on the nonwoven material 200, each of which may reduce curling of the multilayer nonwoven material 200 due to the increased bonding between the two materials. Example positioning, sizing, and orientation of support areas 240 for facilitating reduction of curling, e.g., while the nonwoven material 200 is under tension, are described below, but it will be understood that such positions, sizes, and orientations for support areas 240 are provided by way of example and that other positions, sizes, and orientations for support areas 240 are within the scope of the present disclosure.

In certain example embodiments, at least some of support areas 240 may be positioned adjacent first side portion 225 of first perforated zone 220. The size, position, and/or orientation of the support areas 240 on the nonwoven material 200 adjacent first perforated zone 220 may advantageously limit curling of the nonwoven material 200 while the nonwoven material 200 is under tension. For example, support areas 240 may limit elongation of the nonwoven material 200 due to the presence of apertures 221 within first perforated zone 220 and may thus reduce the risk of web fold-overs and flips during high-speed converting, adhesive overspray, and other product issues while the nonwoven material 200 is under tension.

In some example embodiments, as detailed more specifically below, such support areas 240 can be formed directly into the material 200 itself during a hydroentangling process. As some examples, the support areas 240 can be formed as elongated protrusions, e.g., via a patterning sleeve with a series of grooves that allow fibers from a projection layer to consolidate and form the support areas. However, other methods of forming such support areas 240 are contemplated.

In general, the support areas 240 may be located on side edges of a nonwoven material, such as nonwoven material 200, which may be non-apertured lanes used for attaching (e.g., gluing, bonding, etc.) the nonwoven material 200, or may extend across the whole width of the nonwoven material 200 in other example embodiments. The support areas 240 may be oriented parallel to the cross-machine direction or may be oriented parallel to the machine direction. The support areas 240 may also be orientated at other angles with respect to the machine direction. The support areas 240 may vary in length, width, and depth, and the distance between adjacent support areas 240 may be adjusted also to provide for various levels of web reinforcement. Further details will be described below.

Support areas 240 may be formed on a selected portion of nonwoven material 200. For example, a collective area of support areas 240 on the nonwoven material 200 (e.g., at first surface 211) may be no less than a half percent (0.5%) and no greater than thirty percent (30%), such as no less than five percent (5%) and no greater than twenty percent (20%), of a total area of the nonwoven material 200, e.g., at first surface 211. Thus, support areas 240 may occupy only a portion of nonwoven material 200 while advantageously limiting curling of the nonwoven material 200 while under tension.

Support areas 240 may be sized and/or shaped in a selected manner. For example, support areas 240 may be elongated on nonwoven material 200. Thus, e.g., support areas 240 may be bar or beam shaped in certain example embodiments. Moreover, support areas 240 may be elongated along a length LB of support areas 240, and a width WB of support areas 240 may be defined perpendicular to the length LB of support areas 240, e.g., in the base plane 215 of the first surface 211 of the nonwoven material 200. In example embodiments, the length LB of each support area 240 may be no less than three times (3×) greater than the width WB of each support area 240, such as no less than five times (5×) greater than the width WB of each support area 240, such as no less than seven times (7×) greater than the width WB of each support area 240. In the example embodiment shown in FIGS. 2 and 3, support areas 240 are elongated along the lateral direction LA. Thus, e.g., the length LB of support areas 240 may be oriented generally parallel to the lateral direction LA, and the width WB of support areas 240 may be oriented generally parallel to the longitudinal direction LO. However, it will be understood that support areas 240 may have other orientations on the nonwoven material 200 in alternative example embodiments. For instance, support areas 240 may be oriented at an angle with respect to one or both of the lateral direction LA and the longitudinal direction LO.

Again, the support areas 240 may be configured in a variety of shapes and sizes. In some example embodiments, such as where the support areas 240 are formed through hydroentangling, the support areas 240 may be generally semi-cylindrical in shape. In other example embodiments, the support areas 240 may have a triangular prism shape, a rectangular prism shape, etc. In certain example embodiments, the support areas 240 may be configured to not include any openings or apertures. Although, support areas 240 may be flush with base plane 215 at a proximal end portion 244 of support areas 240. Moreover, support areas 240 may be generally filled with fibers between distal end portion 246 and proximal end portion 244 of support area 240—particularly where support areas 240 are formed by fluid-entangling. Moreover, support areas 240 may be filled by the fibers of nonwoven material 200 between first and second surfaces 211, 213 of the nonwoven material 200. Even more particularly, support areas 240 may preferentially be filled with fibers of one of the layers of the nonwoven material 200 where material 200 is a laminate material.

In certain example embodiments, such as where the support areas 240 are disposed on an apertured material, such as nonwoven material 200, the length LB of the support areas 240 (as measured between opposite ends of the support area 240) may extend for a length between the edge of the material 200 and the first feature zone of the material 200 of between about 25% and about 100%, or between about 33% and 100%, or between about 50% and 100%, or between about 66% and 100% of a total length between edge of the material and the first feature zone. In more particular embodiments, the length LB may advantageously be between about five millimeters (5 mm) and about four hundred millimeters (400 mm). Moreover, in example embodiments and depending upon the arrangement of support areas 240 on the nonwoven material 200, the length LB of the support areas 240 may be between about two hundred and fifty millimeters (250 mm) and about four hundred millimeters (400 mm), between about one hundred and fifty millimeters (150 mm) and about three hundred millimeters (300 mm), between about fifty millimeters (50 mm) and about one hundred millimeters (100 mm), or between about twelve millimeters (12 mm) and about fifty millimeters (50 mm). In certain example embodiments, the width WB of the support areas 240 (as measured between opposite sides of the support area 240) may be between about a half millimeter (0.5 mm) and about six millimeters (6 mm), such as between about one millimeter (1 mm) and about three millimeters (3 mm), such as about two millimeters (2 mm). In certain example embodiments, the support areas 240 may have a height HB (as measured in a direction perpendicular to the base plane 215, e.g., from base plane 215 to a distal end portion 246 of support area 240) of between about a half millimeter (0.5 mm) to about twelve millimeters (12 mm), such as from about one millimeter (1 mm) to about five millimeters (5 mm), such as from about one and one-tenth millimeter (1.1 mm) to about three millimeters (3 mm), such as about two millimeters (2 mm). In certain example embodiments, the average height of the support areas 240 may be less than an average height of the nodes 222. For instance, the average height of the support areas 240 may be no less than two percent (2%) and no greater than seventy percent (70%), such as no less than five percent (5%) and no greater than fifty percent (50%), such as no less than ten percent (10%) and no greater than forty percent (40%), of the average height of the nodes 222. Where the nonwoven material 200 includes both nodes 222 and support areas 240, the support areas 240 may extend away from the base plane 215 in the same direction as the nodes 222. In alternative example embodiments, the support areas 240 may extend away from the base plane 215 in the opposite direction as the nodes 222.

As may be seen in FIG. 2, support areas 240 may be positioned at first side portion 216 of nonwoven material 200. Moreover, support areas 240 may be positioned between a first side portion 225 of first perforated zone 220 and first side portion 216 of nonwoven material 200, e.g., along the lateral direction LA. Moreover, support areas 240 may be in a non-apertured lane between first side portion 225 of first perforated zone 220 and first side portion 216 of nonwoven material 200, e.g., that is used for attaching (e.g., gluing, bonding, etc.) the nonwoven material 200. Thus, e.g., support areas 240 may extend along the lateral direction LA between first side portion 225 of first perforated zone 220 and first side portion 216 of nonwoven material 200. Support areas 240 may also be distributed along the longitudinal direction LO. For instance, support areas 240 may be spaced apart along the longitudinal direction LO at first side portion 216 of nonwoven material 200 in certain example embodiments. In other example embodiments, support areas 240 may be spaced apart along the longitudinal direction LO at first side portion 225 of first perforated zone 220, e.g., such that support areas 240 are not located adjacent second perforated zone 230 or other portions of the nonwoven material 200. Adjacent support areas 240 may be spaced apart by no less than about ten millimeters (10 mm) and no greater than about two hundred millimeters (200 mm), such as no less than about fifteen millimeters (15 mm) and no greater than about one hundred millimeters (100 mm), such as about twenty millimeters (20 mm), in certain example embodiments. Support areas 240 may be arranged parallel to one another in certain example embodiments. Support areas 240 may additionally be spaced apart uniformly in the longitudinal direction LO, but this is not required in all example embodiments.

Nonwoven material 200 may also include a plurality of additional support areas 242, e.g., formed in the same or similar manner that described above for support areas 240. Additional support areas 242 may be positioned opposite support areas 240 on nonwoven material 200. Thus, e.g., additional support areas 242 may be positioned at second side portion 218 of nonwoven material 200. Moreover, additional support areas 242 may be positioned between a second side portion 226 of first perforated zone 220 and second side portion 218 of nonwoven material 200, e.g., along the lateral direction LA. For instance, additional support areas 242 may be in a non-apertured lane between second side portion 226 of first perforated zone 220 and second side portion 218 of nonwoven material 200, e.g., that is used for attaching (e.g., gluing, bonding, etc.) the nonwoven material 200. Additional support areas 242 may extend along the lateral direction LA between second side portion 226 of first perforated zone 220 and second side portion 218 of nonwoven material 200. Additional support areas 242 may also be distributed along the longitudinal direction LO. For instance, additional support areas 242 may be spaced apart along the longitudinal direction LO at second side portion 218 of nonwoven material 200 in certain example embodiments. In other example embodiments, additional support areas 242 may be spaced apart along the longitudinal direction LO at second side portion 226 of first perforated zone 220, e.g., such that support areas 240 are not located adjacent second perforated zone 230 or other portions of the nonwoven material 200. In at least some example embodiments, the support areas 240 and the support areas 242 may be aligned along the lateral direction LA.

The positioning of support areas 240 and additional support areas 242 on nonwoven material 200 may be complementary to each other. Thus, e.g., support areas 240 may limit curling of the nonwoven material 200 at the first side portion 216 of nonwoven material 200, and the additional support areas 242 may limit curling of the nonwoven material 200 at the second side portion 218 of nonwoven material 200.

The size, position, and/or orientation of support areas 240 on the nonwoven material 200 shown in FIGS. 2 and 3 is provided by way of example. It will be understood that other sizes, positions, and/or orientations of support areas 240 on the nonwoven material 200 are within the scope of the present disclosure.

Although discussed in the context of being formed through as part of a hydroentangling process above, support areas 240 contemplated within the scope of this disclosure can be formed according to different processes. In further example embodiments, support areas 240 may be formed as bonds—formed through heat, ultrasonic, and/or pressure, or other bonding methods known in the art. Alternatively, support areas 240 may be embossments within the nonwoven material 200 forming discrete, densified regions. Such alternative formation processes can likewise result in a nonwoven material 200 with increased resistance to elongation under tension, helping to solve the disclosed converting challenges. As would be understood, the specific characteristics of such support areas 240 would differ according to the different formation methods. For example, the specific densities, caliper, and/or the fiber-to-fiber bonding may be different between support areas 240 formed by different methods. Further, the different methodologies may result in different shapes for the support areas 240—for instance, forming the support areas 240 through a bonding process may result in flat structures, as opposed to protruding structures formed according to a hydroentangling process. However, the preferred general longitudinal and lateral direction dimensions of such support areas 240 described above with respect to support areas 240 formed according to hydroentangling processes are equally applicable to support areas 240 formed according to other methods.

Although the support areas 240 may be most advantageously useful within example embodiments of nonwoven material 200 including feature zones with apertures and nodes, it should be understood that such support areas 240 according to example aspects of the present disclosure are useful in other contexts as well. For example, nonwoven materials including laminates of materials having differing mechanical properties can cause undesired curling or folding or other effects when placed under tension. Accordingly, example embodiments according to the present disclosure may include nonwoven materials 200 including laminates of two different webs having differing mechanical properties which do not include any nodes 222, apertures 221, or ligaments 223 but do include such support areas 240. Further example embodiments according to aspects of the present disclosure include nonwoven materials 200 including laminates of two different webs having differing mechanical properties which do not include any nodes 222 or ligaments 223, but do include apertures 221 and such support areas 240. Such contemplated example embodiments include materials 200 where the nodes 222 or apertures 221 are arranged in configurations as described with respect to the feature zones 21, 25 and/or the patterns of nodes 222 or apertures 221 throughout this disclosure.

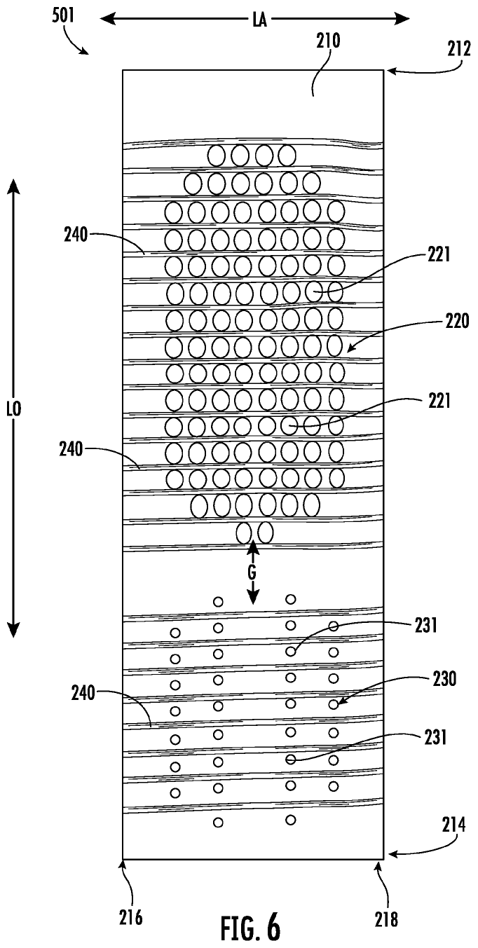

Turning to FIG. 5, depicting a nonwoven material 501 according to another example embodiment of the present subject matter, support areas 240 may be elongated along the lateral direction LA. Moreover, support areas 240 may extend between first and second side portions 216, 218 of the nonwoven material 501 along the lateral direction LA. Accordingly, support areas 240 may extend across first perforated zone 220 from first side portion 216 of the nonwoven material 501 to the second side portion 218 of the nonwoven material 501 along the lateral direction LA. Similarly, support areas 240 may extend across second perforated zone 230 from first side portion 216 of the nonwoven material 501 to the second side portion 218 of the nonwoven material 501 along the lateral direction LA. Conversely, support areas 240 may not be position at the gap G between first and second perforated zones 220, 230 and/or at other unperforated zones of the nonwoven material 501 in certain example embodiments.

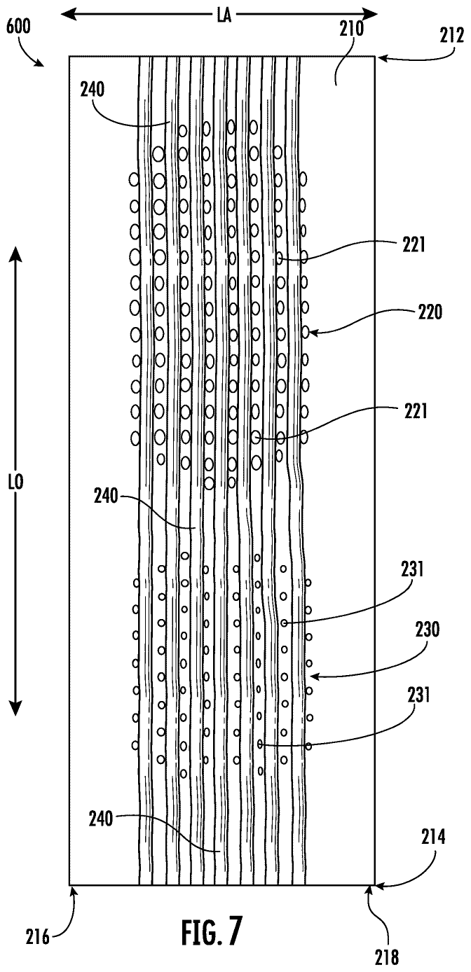

As shown in FIG. 6, on a nonwoven material 600 according to another example embodiment of the present subject matter, support areas 240 may be elongated along the longitudinal direction LO. Moreover, support areas 240 may extend between first and second end portions 212, 214 of the nonwoven material 501 along the longitudinal direction LO. Accordingly, support areas 240 may extend across first and second perforated zones 220, 230 from first end portion 212 of the nonwoven material 501 to the second end portion 214 of the nonwoven material 501 along the longitudinal direction LO. Support areas 240 may also extend across the gap G between first and second perforated zones 220, 230 and across the unperforated zones the nonwoven material 501 at the first and second end portions 212, 214 of the nonwoven material 501; however, support areas 240 may not be positioned adjacent first and second side portions 216, 218 of the nonwoven material 501. In the example embodiment shown in FIG. 6, support areas 240 may also be distributed along the lateral direction LA. For instance, support areas 240 may be spaced apart along the lateral direction LA at first and second perforated zones 220, 230 in certain example embodiments.

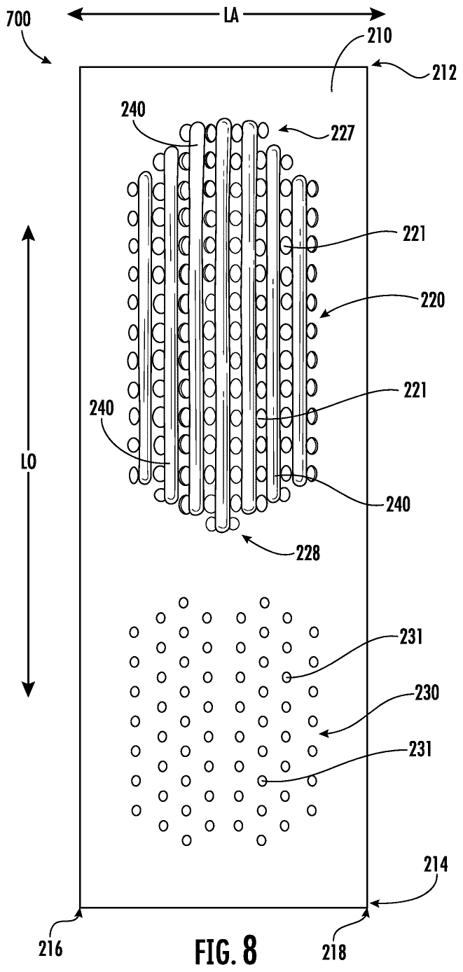

Turning to FIG. 7, on a nonwoven material 700 according to another example embodiment of the present subject matter, support areas 240 may be elongated along the longitudinal direction LO. Moreover, support areas 240 may extend between first and second end portions 227, 228 of the first perforated zone 220 along the longitudinal direction LO. Accordingly, support areas 240 may extend across first perforated zone 220 from first end portion 227 of the first perforated zone 220 to the second end portion 228 of the first perforated zone 220 along the longitudinal direction LO. Conversely, support areas 240 may not be positioned at second perforated zone 230, the gap G between first and second perforated zones 220, 230 and/or at other unperforated zones of the nonwoven material 501 in certain example embodiments. In the example embodiment shown in FIG. 7, support areas 240 may also be distributed along the lateral direction LA. For instance, support areas 240 may be spaced apart along the lateral direction LA at first perforated zone 220 in certain example embodiments.

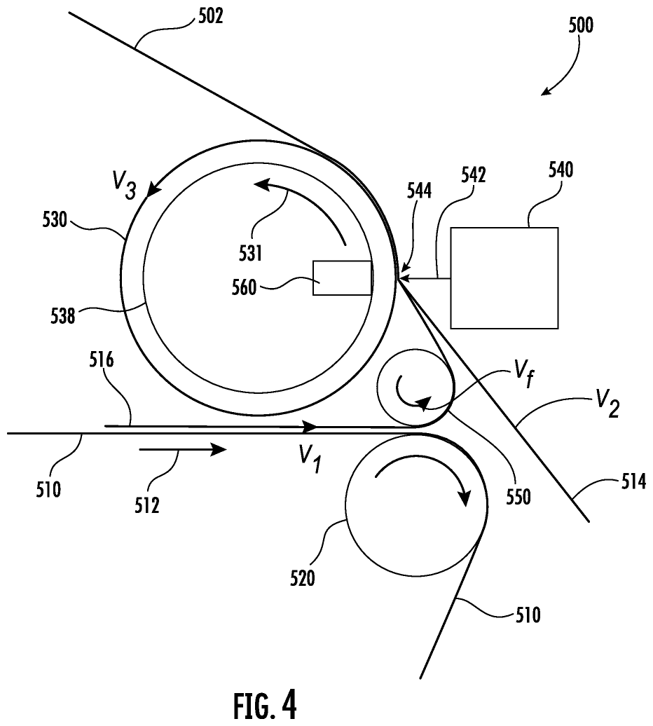

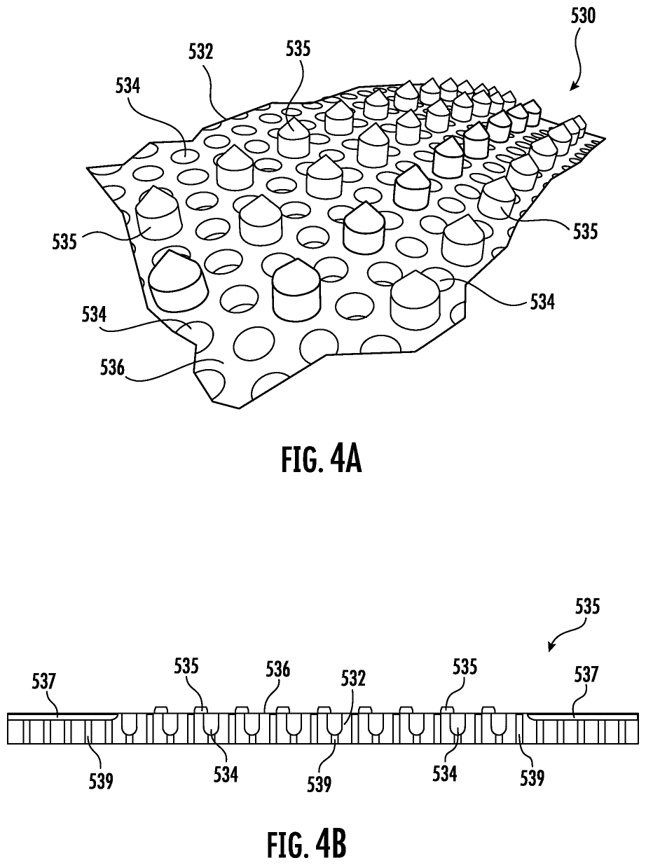

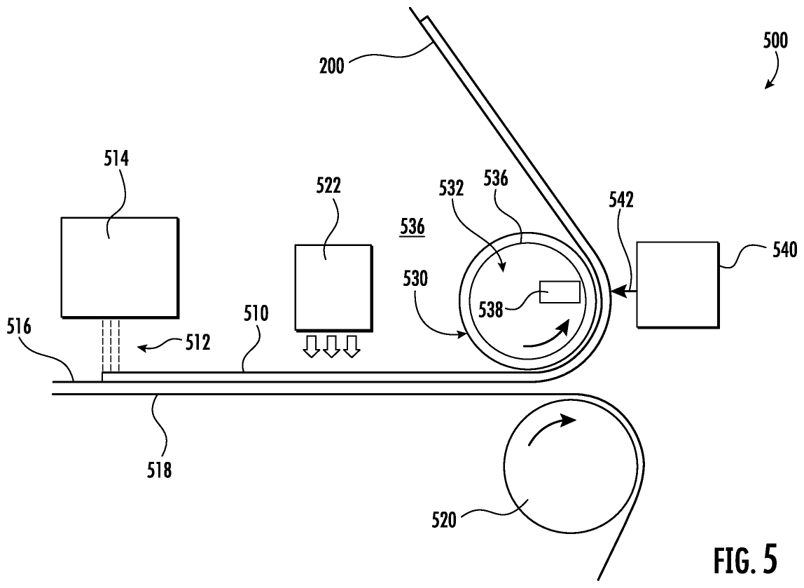

Referring to FIG. 4 there is shown a first example embodiment of a process and apparatus 500 for forming a fluid-entangled laminate web 502, such as nonwoven material 200, e.g., with apertures 221, nodes 222, and/or support areas 240, according to example aspects of the present invention. In some example embodiments, the process and apparatus 500 may be the same or similar to those described with respect to the processes and apparatuses in U.S. Pat. No. 11,365,495, titled “Process for making fluid-entangled laminate webs with hollow projections and apertures”, to Mark J. Beitz et al., the entirety of which is hereby incorporated by reference.

The process and apparatus 500 may include entangling and hence bonding a support layer or web 514 and a projection layer or web 516. In some example embodiments, the fibers of the support web 514 may be similar to those described with respect to the support web 14 in U.S. Pat. No. 9,327,473, titled “Fluid-entangled laminate webs having hollow projections and a process and apparatus for making the same”, to Niall Finn et al., the entirety of which is hereby incorporated by reference. Similarly, the fibers of the projection web 516 may be similar to those described with respect to the projection web 16 in U.S. Pat. No. 9,327,473, the entirety of which is hereby incorporated by reference.

Suitable materials for the support layer 514 can include a fibrous nonwoven web made from a plurality of randomly deposited fibers which may be staple length fibers such as are used, for example, in carded webs, air laid webs, etc., or the materials may be more continuous fibers such as are found in, for example, meltblown or spunbond webs. Due to the functions the support layer 514 must perform, the support layer 514 may have a higher degree of integrity than the projection web 516. In this regard, the support layer 514 may be able to remain substantially intact when the support layer 514 is subjected to the fluid-entangling process discussed in greater detail below. The degree of integrity of the support layer 514 may be such that the material forming the support layer 514 resists being driven down into and filling the hollow nodes 222 of the projection web 516 and support areas 240, for example by resisting being driven into and filling the forming holes 534 and the forming indentations 537 of the forming surface 530 during formation of the web 502. As a result, when the support layer 514 is a fibrous nonwoven web, it may be desirable that the support layer 514 has a higher degree of fiber-to-fiber bonding and/or fiber entanglement than the fibers in the projection web 516. While it is desirable to have fibers from the support layer 514 entangle with the fibers of the projection web 516 adjacent the interface between the two layers, it is generally desired that the fibers of this support layer 514 not be integrated or entangled into the projection web 516 to such a degree that large portions of these fibers find their way inside the hollow nodes 222.

The type, basis weight, strength and other properties of the support layer 514 may be chosen and varied depending upon the particular end use of the resultant laminate 502. When the laminate 502 is to be used as part of an absorbent article such as a personal care absorbent article, wipe, etc., it is may be generally desirable that the support layer 514 be a layer that is fluid pervious, has good wet and dry strength, is able to absorb fluids such as body exudates, possibly retain the fluids for a certain period of time and then release the fluids to one or more subjacent layers. In this regard, fibrous nonwovens, such as spunbond webs, meltblown webs and carded webs, such as airlaid webs, bonded carded webs, and coform materials, are particularly well-suited as support layers 514. Foam materials and scrim materials are also well-suited. In addition, the support layer 514 may be a multi-layered material due to the use of several layers or the use of multi-bank formation processes as are commonly used in making spunbond webs and meltblown webs as well as layered combinations of meltblown and spunbond webs. In the formation of such support layers 514, both natural and synthetic materials may be used alone or in combination to fabricate the material. Generally, for the end-use applications outlined herein, support layer 514 basis weights may range between about five grams per square meter (5 gsm) and about forty grams per square meter (40 gsm) though basis weights outside this range may be used depending upon the particular end-use application.

The type, basis weight and porosity of the support web 514 may affect the process conditions necessary to form the nodes 222 in the projection web 516 and the support areas 240 between support web 514 and projection web 516. Heavier basis weight materials may increase the entangling force of the entangling fluid streams needed to form the nodes 222 in the projection web 516 and the support areas 240 between support web 514 and projection web 516. However, heavier basis weight support layers 514 may also provide improved support for the projection web 516 as a major problem with the projection web 516 by itself is that the projection web 516 is too stretchy to maintain the shape of the nodes 222 post the formation process. The projection web 516 by itself unduly elongates in the machine direction due to the mechanical forces exerted on the projection web 516 by subsequent winding and converting processes which diminish and distort the nodes 222. Also, without the support layer 514, the nodes 222 in the projection web 516 collapse due to the winding pressures and compressive weights the projection web 516 experiences in the winding process and subsequent conversion and do not recover to the extent they do with the support layer 514. Such material property differences may also be mitigated by support areas 240, as described above. For instance, the support areas 240 between support web 514 and projection web 516, e.g., and the resultant increase in the bonding or entanglement of fibers of the projection web 516 at the discrete support areas 240 and between fibers of the support web 514 and fibers of the projection web 516 at the support areas 240, may advantageously limit the elongation differential between the support web 514 and the projection web 516 under tension, and thereby reduce curling of the laminate web 502 under tension, particularly when the laminate web 502 includes apertures 221.

The projection web 516 may be made from a plurality of randomly deposited fibers, which may be staple length fibers such as those that are used, for example, in carded webs, airlaid webs, coform webs, etc. or the fibers may be more continuous fibers such as those that are found in, for example, meltblown or spunbond webs. The fibers in the projection web 516 desirably may have less fiber-to-fiber bonding and/or fiber entanglement and thus less integrity as compared to the integrity of the support layer 514, especially when the support layer 514 is a fibrous nonwoven web. The fibers in the projection web 516 may have no initial fiber-to-fiber bonding for purposes of allowing the formation of the hollow nodes 222 as will be explained in further detail below in connection with the description of the process and apparatus for forming the fluid-entangled laminate web 502. Alternatively, when both the support layer 514 and the projection web 516 are both fibrous nonwoven webs, the projection web 516 may have less integrity than the support web 514 due to the projection web 516 having, for example, less fiber-to-fiber bonding in areas, such as nodes 222 and with apertures 221, less adhesive, or less pre-entanglement of the fibers forming the web 516.

The projection web 516 may have a sufficient amount of fiber movement capability to allow the below-described fluid entangling process to be able to move fibers of the projection web 516 out of the X-Y plane of the projection web 516 and into the perpendicular or Z-direction of the web 516 (the direction of the thickness of web 516) so as to be able to form hollow nodes 222 and/or support areas 240. If more continuous fiber structures are being used such as meltblown or spunbond webs, it is desirable to have little or no pre-bonding of the projection web 516 prior to the fluid entanglement process. Longer fibers such as are generated in meltblowing and spunbonding processes (which are often referred to as continuous fibers to differentiate such fibers from staple length fibers) may typically require more force to displace the fibers in the Z-direction than will shorter, staple length fibers that typically have fiber lengths less than one hundred millimeters (100 mm) and more typically fiber lengths in the ten millimeters (10 mm) to sixty millimeters (60 mm) range. Conversely, staple fiber webs, such as carded webs and airlaid webs, can have some degree of pre-bonding or entanglement of the fibers due to the shorter length of such staple fibers. Such shorter fibers may require less fluid force from the fluid entangling streams to move them in the Z-direction to form the hollow nodes 222 and/or support areas 240. As a result, a balance may be selected between fiber denier, fiber length, degree of pre-fiber bonding, fluid force, web speed, and dwell time so as to be able to create the hollow nodes 222 and/or support areas 240 without, unless desired, forming apertures in land areas or forcing too much material into the interior of the nodes 222 thereby making the nodes 222 too rigid for some end-use applications.

Generally, the projection web 516 may have a basis weight ranging between about ten grams per square meter (10 gsm) and about eighty-five grams per square meter (85 gsm) for the uses outlined herein but basis weights outside this range may be used depending upon the particular end-use application. Spunbond webs may typically have basis weights of between about fifteen grams per square meter (15 gsm) and about fifty grams per square meter (50 gsm) when being used as the projection web 516. Fiber diameters may range between about five microns (5 μm) and about twenty microns (20 μm). The fibers may be single component fibers formed from a single polymer composition, or the fibers may be bicomponent or multicomponent fibers wherein one portion of the fiber has a lower melting point than the other components so as to allow fiber-to-fiber bonding through the use of heat and/or pressure. Hollow fibers may also be used. The fibers may be formed from any polymer formulations typically used to form spunbond webs. Examples of such polymers include, but are not limited to, polypropylene (PP), polyester (PET), polyamide (PA), polyethylene (PE) and polylactic acid (PLA). The spunbond webs may be subjected to post-formation bonding and entangling techniques if necessary to improve the processability of the web prior to it being subjected to the projection forming process.

Meltblown webs may typically have basis weights of between about twenty grams per square meter (20 gsm) and about fifty grams per square meter (50 gsm) when being used as the projection web 516. Fiber diameters may range between about a half micron (0.5 μm) and about five microns (5 μm). The fibers may be single component fibers formed from a single polymer composition or they may be bicomponent or multicomponent fibers wherein one portion of the fiber has a lower melting point than the other components so as to allow fiber-to-fiber bonding through the use of heat and/or pressure. The fibers may be formed from any polymer formulations typically used to form the aforementioned spunbond webs. Examples of such polymers include, but are not limited to, PP, PET, PA, PE and PLA.

Carded and airlaid webs use staple fibers that may typically range in length between about ten millimeters (10 mm) and about one hundred millimeters (100 mm). Fiber denier will range between about 0.5 denier and about 6 denier depending upon the particular end use. Basis weights may range between about twenty grams per square meter (20 gsm) and about eighty-five grams per square meter (85 gsm). The staple fibers may be made from a wide variety of polymers including, but not limited to, PP, PET, PA, PLA, cotton, rayon flax, wool, hemp and regenerated cellulose such as, for example, viscose. Blends of fibers may be utilized too such as blends of bicomponent fibers and single component fibers as well as blends of solid fibers and hollow fibers. If bonding is desired, it may be accomplished in a number of ways including, for example, through-air bonding, calender bonding, point bonding, chemical bonding and adhesive bonding such as powder bonding. If needed, to further enhance the integrity and processability of such webs prior to the projection forming process, the fibers may be subjected to pre-entanglement processes to increase fiber entanglement within the projection web 516 prior to the formation of the nodes 222 and/or support areas 240. Hydroentangling is particularly advantageous in this regard.

While the foregoing nonwoven web types and formation processes are suitable for use in conjunction with the projection web 516, it is anticipated that other webs and formation processes may also be used provided the webs are capable of forming the hollow nodes 222 and/or support areas 240.

As shown in FIG. 4, the apparatus 500 includes a first transport belt 510, a transport belt drive roll 520, a projection forming surface 530, a fluid entangling device 540, an optional overfeed roll 550, and a fluid removal system 560, such as a vacuum or other conventional suction device. Such vacuum devices and other mechanisms are well known to those of ordinary skill in the art. The transport belt 510 is used to carry the projection web 516 into the apparatus 500. If any pre-entangling is to be done on the projection web 516 upstream of the process 500 shown in FIG. 4, the transport belt 510 may be porous. The transport belt 510 travels in a first direction (which is the machine direction) as shown by arrow 512 at a first speed or velocity V₁. The transport belt 510 may be driven by the transport belt drive roller 520 or other suitable mechanism as are well known to those of ordinary skill in the art.

The projection forming surface 530 as shown in FIG. 4 is in the form of a texturizing drum 530, a partially exploded view of the surface which is shown in FIG. 4A and a partial, section view of which is shown in FIG. 4B. The projection forming surface 530 moves in the machine direction as shown by arrow 531 in FIG. 4 at a speed or velocity V₃. The projection forming surface 530 is driven and speed controlled by any suitable drive mechanism (not shown), such as electric motors and gearing as are well known to those of ordinary skill in the art. The texturing drum 530 depicted in FIGS. 4A and 4B includes a forming surface 532 containing a pattern of forming holes 534 that correspond to the shape and pattern of the desired nodes 222 in the projection web 516. The forming holes 534 are separated by a land area 536. The forming holes 534 may be of any shape and any pattern. As may be seen from FIGS. 2, 3, and 5 through 7 depicting the laminate web 502 (nonwoven material 200) according to example aspects of the present invention, the hole shapes are round, but it should be understood that any number of shapes and combination of shapes may be used depending on the end use application. Examples of possible hole shapes include, but are not limited to, ovals, crosses, squares, rectangles, diamonds, hexagons and other polygons. Such shapes may be formed in the projection forming surface 530 by casting, punching, stamping, laser-cutting and water-jet cutting. The spacing of the forming holes 534 and therefore the degree of land area 536 may also be varied depending upon the particular end application of the fluid-entangled laminate web 502. Further, the pattern of the forming holes 534 in the texturizing drum 530 may be varied depending upon the particular end application of the fluid-entangled laminate web 502. The material forming the texturizing drum 530 may be any number of suitable materials commonly used for such forming drums, including, but not limited to, sheet metal, plastics and other polymer materials, rubber, etc. The forming holes 534 may be formed in a sheet of the material 532 that is then formed into a texturizing drum 530 or the texturizing drum 530 may be molded or cast from suitable materials or printed with 3D printing technology.

The forming surface 532 also includes a pattern of protrusions 535 that correspond to the shape and pattern of the desired apertures 221 in the laminate web 502. The protrusions 535 are adjacent land area 536. The protrusions 535 may be of any shape and any pattern. As may be seen from FIGS. 2, 3, and 5 through 6 depicting the laminates 502 (nonwoven material 200) according to example aspects of the present invention, the protrusions are “spike-shaped”/pointed to produce round apertures, but it should be understood that any number of shapes and combination of shapes may be used depending on the end use application of the laminate web 502. Examples of possible protrusion shapes include, but are not limited to, pointed, domed and flat; the protrusions 535 may be further configured to produce apertures 221 having any one of the following shapes: ovals, crosses, squares, rectangles, slots, knife edges, diamond shapes, hexagons, and other polygons. The shape of the protrusion 535 may be varied, so long as the selected shape results in a protrusion 535 that is capable of moving the fibers of the support layer 514 and of the projection web 516 to form an aperture 221 in the laminate web 502 using the energy from the entangling fluid 542 coming out of the projection fluid jets. The apertures 221 are formed at the contact points that the laminate web 502 has with the protrusions 535. As the protrusions 535 move the fibers of the laminate web 502 as the laminate web 502 passes over the projection forming surface 530, the fibers of the laminate web 502 (fibers from the support layer 514 and the projection web 516) gather at the base perimeter of the protrusion 535. Fibers from the support layer 514 become entangled with fibers from the projection web 516 around the circumference/perimeter of the aperture 221. Therefore, placement of the protrusions 535 on the projection forming surface 530 does not cause the solid surface area 536 to go away. The protrusions 535 may be formed in the drum surface by casting, threaded attachment, press-fit, weld attachment, machining, grinding, punching, or stamping. Protrusions 535 may also be secured to projection forming surface 530 with screws, bolts, rivets, compression fittings, weld attachment, adhesive attachment, or other mechanical mechanisms. The spacing of the protrusions 535 and, therefore, the degree of land area 536 may also be varied depending upon the particular end application of the fluid-entangled laminate web 502. Further, the pattern of the protrusions 535 in the projection forming surface 530 may be varied depending upon the particular end application of the fluid-entangled laminate web 502. The protrusions 535 may be formed in a sheet of the material 532 that is then formed into the projection forming surface 530 or the projection forming surface 530 may be molded or cast from suitable materials or printed with 3D printing technology.

The forming surface 532 also includes a pattern of forming sockets or forming indentions 537 that correspond to the shape and pattern of the desired support areas 240 between the support layer 514 and the projection web 516. The forming indentions 537 may be of any shape and any pattern. As may be seen from FIGS. 2, 3, and 5 through 7 depicting the laminate web 502 (nonwoven material 200) according to example aspects of the present invention, the forming indentions 537 are trough shaped or elongated, but it should be understood that any number of shapes and combination of shapes may be used depending on the end use application. Examples of possible forming indention shapes include, but are not limited to, circles, ovals, crosses, squares, rectangles, diamonds, hexagons and other polygons. Such shapes may be formed in the projection forming surface 530 by casting, punching, stamping, laser-cutting, machining, and water-jet cutting. The spacing of the forming indentions 537 may also be varied depending upon the particular end application of the fluid-entangled laminate web 502. Further, the pattern of the forming indentions 537 in the texturizing drum 530 may be varied depending upon the particular end application of the fluid-entangled laminate web 502.

Typically, the perforated drum 530 is removably fitted onto and over an optional porous inner drum shell 538 so that different forming surfaces 532 may be used for different end product designs. The porous inner drum shell 538 interfaces with the fluid removal system 560 which facilitates pulling the entangling fluid and fibers down into the forming holes 534 in the outer texturizing drum surface 532 thereby forming the hollow nodes 222 in the projection web 516 and also facilitates pulling the entangling fluid and fibers down into the forming indentions 537 in the outer texturizing drum surface 532 thereby forming the support areas 240 by increasing entanglement of fibers of the projection web 516 at these discrete locations and increasing entanglement between fibers of the projection web 516 with fibers of the support layer 514. The porous inner drum shell 538 also acts as a barrier to retard further fiber movement down into the fluid removal system 560 and other portions of the equipment thereby reducing fouling of the equipment. The porous inner drum shell 538 rotates in the same direction and at the same speed as the texturizing drum 530. In addition, to further control the height of the nodes 222, the distance between the inner drum shell 538 and the texturizing drum 530 may be varied. Generally, the spacing between the inner surface of projection forming surface 530 and the outer surface of the inner drum shell 538 may range between about zero millimeters (0 mm) and about five millimeters (5 mm). Other ranges may be used depending on the particular end-use application and the desired features of the fluid-entangled laminate web 502.

The depth of the forming holes 534 in the texturizing drum 530 or other projection forming surface 530 may be between one millimeter (1 mm) and ten millimeters (10 mm), such as between around three millimeters (3 mm) and five millimeters (5 mm), to produce nodes 222 with the shape most useful in the expected common applications. The hole cross-section size may be between about two millimeters (2 mm) and ten millimeters (10 mm), such as between three millimeters (3 mm) and six millimeters (6 mm), as measured along the major axis, and the spacing of the forming holes 534 on a center-to-center basis may be between three millimeters (3 mm) and ten millimeters (10 mm), such as between four millimeters (4 mm) and seven millimeters (7 mm). The pattern of the spacing between forming holes 534 may be varied and selected depending upon the particular end use. Some examples of patterns include, but are not limited to, aligned patterns of rows and/or columns, skewed patterns, hexagonal patterns, wavy patterns and patterns depicting pictures, figures and objects.

The cross-sectional dimensions of the forming holes 534 and the depth of the forming holes 534 influence the cross-section and height of the nodes 222 produced in the projection web 516. Generally, hole shapes with sharp or narrow corners at the leading edge of the forming holes 534 as viewed in the machine direction 531 should be avoided as such sharp or narrow corners can sometimes impair the ability to safely remove the fluid-entangled laminate web 502 from the forming surface 532 without damage to the nodes 222. In addition, the thickness/hole depth in the texturizing drum 530 may generally tend to correspond to the depth or height of the hollow nodes 222. It should be noted, however, that each of the hole depth, spacing, size, shape and other parameters may be varied independently of one another and may be varied based upon the particular end use of the fluid-entangled laminate web 502 being formed.

The depth of the forming indentions 537, e.g., into the forming surface 532, may be less than a depth of the forming holes 534. For instance, as shown in FIG. 4B, in certain example embodiments, the forming holes 534 may be blind holes that extend into the forming surface 532 such that the depth of the forming holes 534 is less than a thickness of the forming surface 532, and the forming indentions 537 may also extend into only a portion of the forming surface 532 such that the depth of the indentions 537 is less than the thickness of the forming surface 532. In other example embodiments, the forming holes 534 may extend through the forming surface 532 such that the depth of the forming holes 534 is equal to a thickness of the forming surface 532; conversely, the forming indentions 537 may extend into only a portion of the forming surface 532 such that the depth of the indentions 537 is less than the thickness of the forming surface 532. The reduced depth of the forming indentions 537 relative to the forming holes 534 may advantageously increase entanglement between the fibers of the projection web 516 to form support areas 240. For instance, fluid impacting and rebounding away from the bottom surface of the forming indentions 537 may move or entangle the fibers of the projection web 516. In general, the depth of the forming holes 534 in the texturizing drum 530 or other projection forming surface 530 may be between a quarter millimeter (0.25 mm) and five millimeters (5 mm), such as between around a half millimeter (0.5 mm) and two millimeters (2 mm), to produce support areas 240 with the desired entanglement of fibers within the projection web 516 and consolidation of fibers of the projection web 516 and fibers of the support layer 514 to produce support areas 240 most useful in the expected common applications. The pattern of the spacing between forming indentions 537 may be varied and selected depending upon the particular end use. Some examples of patterns include, but are not limited to, aligned patterns of rows and/or columns, skewed patterns, hexagonal patterns, wavy patterns and patterns depicting pictures, figures and objects.

The land areas 536 in the forming surface 532 of the texturizing drum 530 are typically solid so as to not pass the entangling fluid 542 emanating from the pressurized fluid jets contained in the fluid entangling devices 540, but, in some instances, it may be desirable to make the land areas 536 fluid permeable to further texturize the exposed surface of the projection web 516 and to assist drainage of fluid, e.g., via drainage holes 539 shown in FIG. 4B. Alternatively, select areas of the forming surface 532 of the texturizing drum 530 may be fluid pervious and other areas impervious. For example, a central zone (not shown) of the texturizing drum 530 may be fluid pervious while lateral regions (not shown) on either side of the central region may be fluid impervious.

The height of the protrusions 535 in the texturizing drum 530 or other projection forming surface 530 may be between one millimeter (1 mm) and ten millimeters (10 mm), such as between around three millimeters (3 mm) and five millimeters (5 mm), to produce apertures 221 that are fully-formed through the laminate web 502. The protrusion cross-section size may be between about two millimeters (2 mm) and ten millimeters (10 mm), such as between three millimeters (3 mm) and six millimeters (6 mm), as measured along the major axis. The spacing between protrusions 535 on the forming surface 532 may be selected based on the location and/or pattern of apertures 221 desired in the laminate web 502. The spacing between protrusions 535 may be selected based on the desired registration with the forming holes 534. In one example aspect, the spacing of the protrusions 535 on a center-to-center basis may be between three millimeters (3 mm) and one hundred millimeters (100 mm). In another example aspect, the spacing of the protrusions 535 may be between five millimeters (5 mm) and thirty millimeters (30 mm) on a center-to-center basis. In a further example aspect, the pattern of spacing between protrusions 535 may be non-uniform such that there is a higher density of protrusions 535 in one area/location of the forming surface 532 than in a neighboring area/location. The neighboring areas/locations on the forming surface 532 may be around the circumference of the forming surface 532 or across the width of the forming surface 532. In a representative example aspect, the protrusions 535 are arranged in “array lanes”; an “array lane” is a pattern of protrusions 535 that may extend across the width of the forming surface 532. A group of array lanes may be located in proximity to each other to form a bigger pattern of protrusions 535; the array lanes may be separated from each other by a distance in the circumferential direction of the forming surface 532. An advantage of a non-uniform distribution of protrusions 535 on the forming surface 532 may be to provide areas without apertures 221 in the laminate web 502 to facilitate adhesive bonding of non-apertured areas of the laminate web 502 within an absorbent article to minimize risk of exposed adhesive.

In the example embodiment of the apparatus 500 shown in FIG. 4, the projection forming surface 530 is shown in the form of a texturizing drum. It should be appreciated however that other mechanisms may be used to create the projection forming surface 530. For example, a continuous foraminous belt or wire (not shown) may be used which includes forming holes 534 formed in the belt or wire at appropriate locations. Alternatively, flexible rubberized belts (not shown) which are impervious to the pressurized fluid entangling streams save the forming holes 534 may be used. Such belts and wires are well known to those of ordinary skill in the art as are the mechanisms for driving and controlling the speed of such belts and wires. A texturizing drum 530 may be more advantageous for formation of the fluid-entangled laminate web 502 according to example aspects of the present invention because the texturizing drum 530 may be made with land areas 536 which are smooth and impervious to the entangling fluid 542 and which do not leave a wire weave pattern on an outer surface of the projection web 516 as wire belts tend to do.

An alternative to a forming surface 532 with a hole-depth defining the projection height is a forming surface 532 that is thinner than the desired projection height but which is spaced away from the porous inner drum shell 538 surface on which the forming surface 532 is wrapped. The spacing between the texturizing drum 530 and porous inner drum shell 538 may be achieved by any mechanism that preferably does not otherwise interfere with the process of forming the hollow nodes 222 and withdrawing the entangling fluid 542 from the equipment 540. For example, one mechanism is a hard wire or filament that may be inserted between the outer texturizing drum 530 and the porous inner drum shell 538 as a spacer or wrapped around the inner porous drum shell 538 underneath the texturizing drum 530 to provide the appropriate spacing. A shell depth of the forming surface 532 of less than two millimeters (2 mm) can make it more difficult to remove the projection web 516 and the laminate 502 from the texturizing drum 530 because the fibrous material of the projection web 516 can expand or be moved by entangling fluid 542 flow into the overhanging area beneath the texturizing drum 530, which in turn can distort the resultant fluid-entangled laminate web 502. It has been found, however, that by using a support layer 514 in conjunction with the projection web 516 as part of the formation process, distortion of the resultant two-layer fluid-entangled laminate web 502 can be greatly reduced. Use of the support web 514 generally facilitates cleaner removal of the fluid-entangled laminate web 502 because the less extensible, more dimensionally stable support layer 514 takes the load while the fluid-entangled laminate 502 is removed from the texturizing drum 530. The higher tension that can be applied to the support layer 514, compared to a single projection web 516, means that as the fluid-entangled laminate 502 moves away from the texturizing drum 530, the nodes 222 can exit the forming holes 534 smoothly in a direction roughly perpendicular to the forming surface 532 and co-axially with the forming holes 534 in the texturizing drum 530. In addition, by using the support layer 514, processing speeds may be increased.

To form the nodes 222 in the projection web 516, to laminate the support layer 514 and the projection web 516 together, to form apertures 221 in the laminate web 502, and to form the support areas 240 between the support layer 514 and the projection web 516, one or more fluid entangling devices 540 are spaced above the projection forming surface 530. The most common technology used in this regard is referred to as spunlace or hydroentangling technology, which uses pressurize water as the fluid for entanglement. As an unbonded or relatively unbonded web or webs are fed into a fluid-entangling device 540, a multitude of high-pressure fluid jets (not shown) from one or more fluid entangling devices 540 move the fibers of the webs and the fluid turbulence causes the fibers to entangle. These fluid streams, which in this case are water, can cause the fibers to be further entangled within the individual webs. The streams can also cause fiber movement and entanglement at an interface of two or more webs/layers thereby causing the webs/layers to become joined together. Still further, if the fibers in a web, such as the projection web 516, are loosely held together, the fibers can be driven out of the X-Y plane of the fibers and thus in the Z-direction to form the nodes 222, which are preferably hollow. Depending on the level of entanglement needed, one or a plurality of such fluid entangling devices 540 may be used.

In FIG. 4, a single fluid entangling device 540 is shown. When multiple devices are used, the entangling fluid pressure in each subsequent fluid entangling device 540 is usually higher than the preceding one so that the energy imparted to the webs/layers increases and so the fiber entanglement within or between the webs/layers increases. This reduces disruption of the overall evenness of the areal density of the web/layer by the pressurized fluid jets while achieving the desired level of entanglement and hence bonding of the webs/layers and formation of the nodes 222 and support areas 240. The entangling fluid 542 of the fluid entangling devices 540 emanates from injectors via jet packs or strips (not shown) including of a row or rows of pressurized fluid jets with small apertures of a diameter usually between eight-hundredths of a millimeter (0.08 mm) and fifteen-hundredths of a millimeter (0.15 mm) and spacing of around a half millimeter (0.5 mm) in the cross-machine direction. The pressure in the jets may be between about five (5) bar and about four hundred (400) bar but typically is less than two hundred (200) bar except for heavy fluid-entangled laminate webs 502 and when fibrillation is required. Other jet sizes, spacings, numbers of jets and jet pressures may be used depending upon the particular end application. Such fluid entangling devices 540 are well known to those of ordinary skill in the art and are readily available from such manufactures as Fleissner of Germany and Andritz-Perfojet of France.

The fluid entangling devices 540 will typically have the jet orifices positioned or spaced between about twenty (20) millimeters and about forty (40) millimeters, and more typically between about twenty (20) millimeters and about thirty (30) millimeters, from the projection forming surface 530 though the actual spacing may vary depending on the basis weights of the materials being acted upon, the fluid pressure, the number of individual jets being used, the amount of vacuum being used via the fluid removal system 560 and the speed at which the equipment is being run.

In the example embodiment shown in FIG. 4, the fluid entangling device 540 is a conventional hydroentangling device, the construction and operation of which are well known to those of ordinary skill in the art. See for example U.S. Pat. No. 3,485,706 to Evans, the contents of which is incorporated herein by reference in its entirety for all purposes. Also see, the description of the hydraulic entanglement equipment described by Honeycomb Systems, Inc., Biddeford, Me., in the article entitled “Rotary Hydraulic Entanglement of Nonwovens”, reprinted from INSIGHT '86 INTERNATIONAL ADVANCED FORMING/BONDING Conference, the contents of which is incorporated herein by reference in its entirety for all purposes.

Returning again to FIG. 4, the projection web 516 is fed into the apparatus and process 500 at a speed V₁, the support layer 514 is fed into the apparatus and process 500 at a speed V₂, and the fluid-entangled laminate web 502 exits the apparatus and process 500 at a speed V₃, which is the speed of the projection forming surface 530 and may also be referred to as the projection forming surface speed. As will be explained in greater detail below, these speeds V₁, V₂, and V₃ may be the same as one another or varied to change the formation process and the properties of the resultant fluid-entangled laminate web 502. Feeding both the projection web 516 and the support layer 514 into the process 500 at the same speed (V₁ and V₂) may produce a fluid-entangled laminate web 502 according to example aspects of the present invention with the desired hollow nodes 222. Feeding both the projection web 516 and the support layer 514 into the process at the same speed, which is faster than the machine direction speed (V₃) of the projection forming surface 530, may also form the desired hollow nodes 222.

Also shown in FIG. 4 is an optional overfeed roll 550, which may be driven at a speed or rate V_{f}. The overfeed roll 550 may be run at the same speed as the speed V₁ of the projection web 516, in which case Vᵢ may equal V₁ or V_{f} may be run at a faster rate to tension the projection web 516 upstream of the overfeed roll 550 when overfeed is desired. Over-feed occurs when one or both of the incoming webs/layers (516, 514) are fed onto the projection forming surface 530 at a greater speed than the projection forming surface speed of the projection forming surface 530. It has been found that improved projection formation in the projection web 516 may be affected by feeding the projection web 516 onto the projection forming surface 530 at a higher rate than the incoming speed V₂ of the support layer 514. In addition, however, it has been discovered that improved properties and projection formation may be accomplished by varying the feed rates of the webs/layers (516, 514) and by also using the overfeed roll 550 just upstream of the texturizing drum 530 to supply a greater amount of fiber via the projection web 516 for subsequent movement by the entangling fluid 542 down into the forming holes 534 in the texturizing drum 530. In particular, by overfeeding the projection web 516 onto the texturizing drum 530, improved projection formation may be achieved including increased projection height.

In order to provide an excess of fiber so that the height of the nodes 222 may be increased or maximized and so that fibers at support areas 240 entangle, the projection web 516 may be fed onto the texturing drum 530 at a greater surface speed (V₁) than the texturizing drum 530 is traveling (V₃). Referring to FIG. 4, when overfeed is desired, the projection web 516 is fed onto the texturizing drum 530 at a speed V₁ while the support layer 514 is fed in at a speed V₂ and the texturizing drum 530 is traveling at a speed V₃, which is slower than V₁ and may be equal to V₂. The overfeed percent or ratio, the ratio at which the projection web 516 is fed onto the texturizing drum 530, may be defined as OF=[(V₁/V₃)−1]×100 where V₁ is the input speed of the projection web 516 and V₃ is the output speed of the resultant fluid-entangled laminate web 502 and the speed of the texturizing drum 530. (When the overfeed roll 550 is being used to increase the speed of the incoming material onto the texturizing drum 530, it should be noted that the speed V₁ of the material after the overfeed roll 550 may be faster than the speed V₁ upstream of the overfeed roll 550. In calculating the overfeed ratio, it is this faster speed V_{f} that should be used.) Good formation of the nodes 222 and support areas 240 has been found to occur when the overfeed ratio is between about five percent (5%) and about fifty percent (50%). Note too, that this overfeeding technique and ratio may be used with respect to not just the projection web 516 only but to a combination of the projection web 516 and the support layer 514 as the webs 514, 516 are collectively fed onto the projection forming surface 530.

In order to minimize the length of projection web 516 that is supporting its own weight before being subjected to the entangling fluid 542 and to avoid wrinkling and folding of the projection web 516, the overfeed roll 550 may be used to carry the projection web 516 at speed V₁ to a position close to the texturizing zone 544 on the texturizing drum 530. In the example illustrated in FIG. 4, the overfeed roll 550 is driven off the transport belt 510, but it is also possible to drive the overfeed roll 550 separately so as to not put undue stress on the incoming projection web material 516. The support layer 514 may be fed into the texturizing zone 544 separately from the projection web 516 and at a speed V₂ that may be greater than, equal to or less than the texturizing drum speed V₃ and greater than, equal to or less than the projection web 516 speed V₁. Preferably, the support layer 514 is drawn into the texturizing zone 544 by frictional engagement with the projection web 516 positioned on the texturizing drum 530, and, once on the texturizing drum 530, the support layer 514 has a surface speed close to the speed V₃ of the texturizing drum 530, or the support layer 514 may be positively fed into the texturizing zone 544 at a speed close to the texturizing drum speed of V₃. The texturizing process causes some contraction of the support layer 514 in the machine direction 531. The overfeed of either the support layer 514 or the projection web 516 may be adjusted according to the particular materials and the equipment and conditions being used so that the excess material that is fed into the texturizing zone 544 is used up thereby avoiding any unsightly wrinkling in the resultant fluid-entangled laminate web 502. As a result, the two webs/layers (516, 514) will usually be under some tension at all times despite the overfeeding process. The take-off speed of the fluid-entangled laminate web 502 must be arranged to be to be close to the texturizing drum speed V₃ such that excessive tension is not applied to the fluid-entangled laminate web 502 in removal of the laminate 502 from the texturizing drum 530 as such excessive tension would be detrimental to the clarity and size of the nodes 222.

After the fluid entanglement occurs from the fluid entangling streams 542 by the fluid entanglement device 540, the precursor web 510 becomes a nonwoven web forming the nonwoven material 200 described above that includes support areas 240, e.g., with nodes 222, ligaments 223 interconnecting the nodes 222, and/or apertures 221 as described above. Such nonwoven materials 200 may be devoid of any binders—such as adhesive or the like—and may be held together solely through fiber entanglement. The process and apparatus 500 may further include removing the web of nonwoven material 200 from the forming surface 530 and drying the hydroentangled web to provide a three-dimensional nonwoven material 200. Drying of the nonwoven material 200 may occur through known techniques by one of ordinary skill in the art. In example embodiments where the precursory web includes binder fibers, the drying of the nonwoven material 200 may activate the binder fibers. Activating the binder fibers can assist with the preservation of the three-dimensionality of the nonwoven material 200 by helping to preserve the geometry and height of the support areas 240 that extend away from the base plane 215 on the first surface 211 of the nonwoven material 200 (as depicted in FIGS. 2 and 3).

Where the support areas 240 are formed by methods or mechanisms other than hydroentangling, the dried nonwoven material 200 may not initially have such support areas 240. Further processes (not shown), such as bonding processes or embossing processes, may impart bonds and/or embossments to the dried nonwoven material 200 to form the support areas 240 in the nonwoven material 200.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

### REFERENCE CHARACTERS

10 Absorbent article
11 Chassis
12 Front waist region
14 Rear waist region
16 Crotch region
18 Longitudinal side edge
19 Body facing surface
20 Longitudinal side edge
21 Perforated zone
22 Front waist edge
23 Apertures
24 Rear waist edge
25 Perforated zone
26 Outer cover
27 Apertures
28 Bodyside liner
29 Longitudinal axis
30 Longitudinal direction
31 Lateral axis
32 Lateral direction
34 Absorbent body
36 Longitudinal edge
38 Longitudinal edge
40 First end edge
42 Second end edge
44 Absorbent assembly
45 Body facing surface
50 Containment flap
52 Containment flap
54 Elasticated waist member
56 Body facing surface
60 Leg elastic member
62 Leg elastic member
64 Base portion
64a Proximal end
64b Distal end
66 Projection portion
68 Flap elastic members
70 Active flap elastic region
71 Tack-down region
72 First longitudinal side edge
74 Second longitudinal side edge
84 Tack-down region
86 Elastic members
91 Back fasteners
92 Front fasteners
94 Stretch component
96 Nonwoven carrier or hook base
98 Fastening component
200 Nonwoven material
210 Fibrous web
211 First surface
212 First end portion (web)
213 Second Surface
214 Second end portion (web)
215 Base plane
216 First side portion
218 Second side portion (web)
220 First perforated zone (web)
221 Apertures
222 Nodes
223 Ligaments
225 First side portion (first zone)
226 Second side portion (first zone)
227 First end portion (first zone)
228 Second end portion (first zone)
230 Second perforated zone
231 Apertures
240 Support areas
242 Additional support areas
244 Open end portion
246 Distal end portion
252 Cap (node)
254 Wall (node)
LAA Length of absorbent article
L1 Length of web
WAA Width of absorbent article
W1 Width of web
HB Height (support areas)
LB Length (support areas)
WB Width (support areas)

### EXAMPLE EMBODIMENTS

First example embodiment: A multilayer nonwoven material, comprising: a laminate nonwoven fibrous web defining a lateral direction and a longitudinal direction, the lateral and longitudinal directions being perpendicular, the laminate nonwoven fibrous web comprising a first web and a second web, the laminate nonwoven fibrous web comprising a perforated zone with a plurality of apertures that are distributed across a width of the perforated zone along the lateral direction and across a length of the perforated zone along the longitudinal direction, wherein the laminate nonwoven fibrous web comprises one or more support areas, fibers of the first web entangled within discrete areas of the first web and consolidated with fibers of the second web to a greater extent at the one or more support areas than at the areas of the laminate nonwoven fibrous web adjacent to the one or more support areas, a collective area of the one or more support areas on the laminate nonwoven fibrous web being no less than a half percent and no greater than thirty percent of a total area of the laminate nonwoven fibrous web.

Second example embodiment: The multilayer nonwoven material of the first example embodiment, wherein the one or more support areas differ from the areas of the laminate nonwoven fibrous web adjacent to the one or more support areas by one or more of basis weight, density, caliper, and fiber-to-fiber bonding.

Third example embodiment: The multilayer nonwoven material of either the first example embodiment or the second example embodiment, wherein the collective area of the one or more support areas on the laminate nonwoven fibrous web is no less than five percent and no greater than twenty percent of a total area of the laminate nonwoven fibrous web.

Fourth example embodiment: The multilayer nonwoven material of any one of the first through third example embodiments, wherein a basis weight of the laminate nonwoven fibrous web at the one or more support areas is greater than the basis weight of the laminate nonwoven fibrous web at the areas of the laminate nonwoven fibrous web adjacent to the one or more support areas.

Fifth example embodiment: The multilayer nonwoven material of any one of the first through fourth example embodiments, wherein a caliper of the laminate nonwoven fibrous web at the plurality of support areas is greater than the caliper of the laminate nonwoven fibrous web at the areas of the laminate nonwoven fibrous web adjacent to the one or more support areas.

Sixth example embodiment: The multilayer nonwoven material of any one of the first through fifth example embodiments, wherein the laminate nonwoven fibrous web comprises a first surface and a second surface, the first surface being opposite the second surface on the laminate nonwoven fibrous web, the plurality of apertures extending between the first and second surfaces of the laminate nonwoven fibrous web, the plurality of support areas filled by the fibers of the first web at the first surface and consolidated with the fibers of the second web between the first and second surfaces of the laminate nonwoven fibrous web.

Seventh example embodiment: The multilayer nonwoven material of any one of the first through sixth example embodiments, wherein the laminate nonwoven fibrous web comprises a first surface and a second surface, the first surface being opposite the second surface on the laminate nonwoven fibrous web, the laminate nonwoven fibrous web comprising a plurality of nodes extending away from a base plane on the first surface.

Eighth example embodiment: The multilayer nonwoven material of the seventh example embodiment, wherein the plurality of nodes are hollow.

Nineth example embodiment: The multilayer nonwoven material of either of the seventh example embodiment or the eighth example embodiment, wherein the plurality of nodes extend from the first surface by a height of the plurality of nodes, the one or more support areas extend from the first surface by a height of the one or more support areas, and an average height of the plurality of nodes is greater than an average height of the one or more support areas.

Tenth example embodiment: The multilayer nonwoven material of any one of the seventh through nineth example embodiments, the fibers of the first web are entangled within discrete areas of the first web and consolidated with the fibers of the second web to a greater extent at the one or more support areas than at the plurality of nodes.

Eleventh example embodiment: The multilayer nonwoven material of any one of the first through tenth example embodiments, wherein the at least one support comprises a plurality of support areas, and the plurality of support areas are discrete from one another on the laminate nonwoven fibrous web.

Twelfth example embodiment: The multilayer nonwoven material of the eleventh example embodiment, wherein: the width of the perforated zone along the lateral direction is less than a width of the nonwoven fibrous web along the lateral direction; the plurality of support areas are elongated along the lateral direction; the plurality of support areas comprises a first plurality of support areas and a second plurality of support areas; the first plurality of support areas are positioned between a first side portion of the perforated zone and a first side portion of the nonwoven fibrous web along the lateral direction, the first plurality of support areas distributed along the longitudinal direction; and the second plurality of support areas are positioned between a second side portion of the perforated zone and a second side portion of the nonwoven fibrous web along the lateral direction, the second plurality of support areas distributed along the longitudinal direction.

Thirteenth example embodiment: The multilayer nonwoven material of the twelfth example embodiment, wherein: a first side lane extends between the first side portion of the perforated zone and the first side portion of the nonwoven fibrous web along the lateral direction; a second side lane extends between the second side portion of the perforated zone and the second side portion of the nonwoven fibrous web along the lateral direction; a length of each of the first plurality of support areas along the lateral direction is no less than twenty-five percent and no greater than one hundred percent of a length of the first side lane along the lateral direction; and a length of each of the second plurality of support areas along the lateral direction is no less than twenty-five percent and no greater than one hundred percent of a length of the second side lane along the lateral direction.

Fourteenth example embodiment: The multilayer nonwoven material of any one of the eleventh through thirteenth example embodiments, wherein the plurality of support areas are arranged parallel with adjacent support areas spaced apart by no less than about ten millimeters and no greater than about two hundred millimeters.

Fifteenth example embodiment: The multilayer nonwoven material of any one of the eleventh through fourteenth example embodiments, wherein: the nonwoven fibrous web comprises a first surface and a second surface, the first surface being opposite the second surface on the nonwoven fibrous web; a distal end portion of each of the plurality of support areas is spaced from the first surface of the nonwoven fibrous web by a height of each of the plurality of support areas; and an average height of the plurality of support areas is no less than a half millimeter and no greater than five millimeters.

Sixteenth example embodiment: The multilayer nonwoven material of any one of the eleventh through fifteenth example embodiments, wherein the one or more support areas are formed on the nonwoven fibrous web by a hydroentangling process.

Seventeenth example embodiment: The multilayer nonwoven material of any one of the eleventh through fifteenth example embodiments, wherein the one or more support areas are formed on the nonwoven fibrous web by one or both of a bonding process and an embossment process.

Eighteenth example embodiment: A garment, comprising a layer formed with the nonwoven material of any one of the eleventh through seventeenth example embodiments.

Nineteenth example embodiment: A multilayer nonwoven material, comprising: a laminate nonwoven fibrous web defining a lateral direction and a longitudinal direction, the lateral and longitudinal directions being perpendicular, the laminate nonwoven fibrous web comprising a first web and a second web, the laminate nonwoven fibrous web comprising a perforated zone with a plurality of apertures that are distributed across a width of the perforated zone along the lateral direction and across a length of the perforated zone along the longitudinal direction, wherein the laminate nonwoven fibrous web comprises a first surface and a second surface, the first surface being opposite the second surface on the laminate nonwoven fibrous web, the laminate nonwoven fibrous web comprising a plurality of nodes extending away from a base plane on the first surface, wherein the laminate nonwoven fibrous web comprises one or more support areas, fibers of the first web entangled within discrete areas of the first web and consolidated with fibers of the second web to a greater extent at the one or more support areas than at the areas of the laminate nonwoven fibrous web adjacent to the one or more support areas.

Twentieth example embodiment: The multilayer nonwoven material of the nineteenth example embodiment, wherein the one or more support areas differ from the areas of the laminate nonwoven fibrous web adjacent to the one or more support areas by one or more of basis weight, density, caliper, and fiber-to-fiber bonding.

Twenty-first example embodiment: The multilayer nonwoven material of either of the nineteenth example embodiment or the twentieth example embodiment, wherein a collective area of the one or more support areas on the laminate nonwoven fibrous web is no less than a half percent and no greater than thirty percent of a total area of the laminate nonwoven fibrous web at the first surface.

Twenty-second example embodiment: The multilayer nonwoven material of the twenty-first example embodiment, wherein the collective area of the one or more support areas on the laminate nonwoven fibrous web is no less than five percent and no greater than twenty percent of a total area of the laminate nonwoven fibrous web.

Twenty-third example embodiment: The multilayer nonwoven material of any one of the nineteenth through twenty-second example embodiments, wherein the plurality of nodes are hollow

[symbol]

Twenty-fourth example embodiment: The multilayer nonwoven material of any one of the first through twenty-third example embodiments, wherein the plurality of nodes extend from the first surface by a height of the plurality of nodes, the one or more support areas extend from the first surface by a height of the one or more support areas, and an average height of the plurality of nodes is greater than an average height of the one or more support areas.

Twenty-fifth example embodiment: The multilayer nonwoven material of any one of the nineteenth through twenty-fourth example embodiments, wherein the fibers of the first web are entangled within discrete areas of the first web and consolidated with the fibers of the second web to a greater extent at the one or more support areas than at the plurality of nodes.

Twenty-sixth example embodiment: The multilayer nonwoven material of any one of the nineteenth through twenty-fifth example embodiments, wherein the at least one support comprises a plurality of support areas, and the plurality of support areas are discrete from one another on the laminate nonwoven fibrous web.

Twenty-seventh example embodiment: The multilayer nonwoven material of the twenty-sixth example embodiment, wherein the plurality of support areas are arranged parallel with adjacent support areas spaced apart by no less than about ten millimeters and no greater than about fifty millimeters.

Twenty-eighth example embodiment: The multilayer nonwoven material of either of the twenty-sixth example embodiment or the twenty-seventh example embodiment, wherein: a distal end portion of each of the plurality of support areas is spaced from the first surface of the nonwoven fibrous web by a height of each of the plurality of support areas; and an average height of the plurality of support areas is no less than a half millimeter and no greater than five millimeters.

Twenty-nineth example embodiment: The multilayer nonwoven material of any one of the twenty-sixth through twenty-eight example embodiments, wherein the plurality of apertures provide a percent open area for the perforated zone no less than about ten percent and no greater than about sixty percent.

Thirtieth example embodiment: The multilayer nonwoven material of any one of the twenty-sixth through twenty-nineth example embodiments, wherein an area of each of the plurality of apertures is no less than about eight millimeters squared and no greater than about eighteen millimeters squared.

Thirty-first example embodiment: A garment, comprising a layer formed with the multilayer nonwoven material of any one of the nineteenth through thirty-first example embodiments,

[symbol]

Thirty-second example embodiment: A multilayer nonwoven material, comprising: a laminate nonwoven fibrous web defining a lateral direction and a longitudinal direction, the lateral and longitudinal directions being perpendicular, the laminate nonwoven fibrous web comprising a first web and a second web, the laminate nonwoven fibrous web comprising a perforated zone with a plurality of apertures that are distributed across a width of the perforated zone along the lateral direction and across a length of the perforated zone along the longitudinal direction, wherein the laminate nonwoven fibrous web comprises a first surface and a second surface, the first surface being opposite the second surface on the laminate nonwoven fibrous web, the laminate nonwoven fibrous web comprising a plurality of hollow nodes extending away from a base plane on the first surface, wherein the laminate nonwoven fibrous web comprises one or more support areas, fibers of the first web entangled at the one or more support and consolidated with fibers of the second web to a greater extent at the one or more support areas than at the areas of the laminate nonwoven fibrous web adjacent to the one or more support areas, the plurality of support areas filled by the fibers of the first web at the first surface and consolidated with the fibers of the second web between the first and second surfaces of the laminate nonwoven fibrous web.

Thirty-third example embodiment: The multilayer nonwoven material of the thirty-second example embodiment, wherein the one or more support areas differ from the areas of the laminate nonwoven fibrous web adjacent to the one or more support areas by one or more of basis weight, density, caliper, and fiber-to-fiber bonding.

Thirty-fourth example embodiment: The multilayer nonwoven material of either the thirty-second example embodiment or the thirty-third example embodiment, wherein a collective area of the one or more support areas on the laminate nonwoven fibrous web is no less than a half percent and no greater than thirty percent of a total area of the laminate nonwoven fibrous web at the first surface.

Thirty-fifth example embodiment: The multilayer nonwoven material of the thirty-fourth example embodiment, wherein the collective area of the one or more support areas on the laminate nonwoven fibrous web is no less than five percent and no greater than twenty percent of a total area of the laminate nonwoven fibrous web.

Thirty-sixth example embodiment: The multilayer nonwoven material of any one of the thirty-second through thirty-fifth example embodiments, wherein the plurality of apertures extend between the first and second surfaces of the laminate nonwoven fibrous web.

Thirty-seventh example embodiment: The multilayer nonwoven material of any one of the thirty-second through thirty-sixth example embodiments, wherein the plurality of hollow nodes extend from the first surface by a height of the plurality of hollow nodes, the one or more support areas extend from the first surface by a height of the one or more support areas, and an average height of the plurality of hollow nodes is greater than an average height of the one or more support areas.

Thirty-eighth example embodiment: The multilayer nonwoven material of any one of the thirty-fourth through thirty-seventh example embodiments, wherein the fibers of the first web are entangled with the fibers of the second web to a greater extent at the one or more support areas than at the plurality of hollow nodes.

Thirty-nine example embodiment: The multilayer nonwoven material of any one of the thirty-fourth through thirty-eighth example embodiments, wherein the at least one support comprises a plurality of support areas, and the plurality of support areas are discrete from one another on the laminate nonwoven fibrous web.

Fortieth example embodiment: The multilayer nonwoven material of the thirty-nineth example embodiment, wherein: the width of the perforated zone along the lateral direction is less than a width of the nonwoven fibrous web along the lateral direction; the plurality of support areas are elongated along the lateral direction; the plurality of support areas comprises a first plurality of support areas and a second plurality of support areas; the first plurality of support areas are positioned between a first side portion of the perforated zone and a first side portion of the nonwoven fibrous web along the lateral direction, the first plurality of support areas distributed along the longitudinal direction; and the second plurality of support areas are positioned between a second side portion of the perforated zone and a second side portion of the nonwoven fibrous web along the lateral direction, the second plurality of support areas distributed along the longitudinal direction.

Forty-first example embodiment: The multilayer nonwoven material of either the thirty-nineth example embodiment or the fortieth example embodiment, wherein: a first side lane extends between the first side portion of the perforated zone and the first side portion of the nonwoven fibrous web along the lateral direction; a second side lane extends between the second side portion of the perforated zone and the second side portion of the nonwoven fibrous web along the lateral direction; a length of each of the first plurality of support areas along the lateral direction is no less than twenty-five percent and no greater than one hundred percent of a length of the first side lane along the lateral direction; and a length of each of the second plurality of support areas along the lateral direction is no less than twenty-five percent and no greater than one hundred percent of a length of the second side lane along the lateral direction.

Forty-second example embodiment: The multilayer nonwoven material of any one of the thirty-nineth through forty-first example embodiments wherein the width of the perforated zone along the lateral direction is less than a width of the nonwoven fibrous web along the lateral direction, the plurality of support areas are elongated along the lateral direction and extend between a first side portion of the nonwoven fibrous web and a second side portion of the nonwoven fibrous web along the lateral direction, and the plurality of support areas distributed along the longitudinal direction.

Forty-third example embodiment: The multilayer nonwoven material of any one of the thirty-nineth through forty-second example embodiments, wherein: a distal end portion of each of the plurality of support areas is spaced from the first surface of the nonwoven fibrous web by a height of each of the plurality of support areas; and an average height of the plurality of support areas is no less than a half millimeter and no greater than five millimeters.

Forty-fourth example embodiment: The multilayer nonwoven material of any one of the thirty-second through forty-third example embodiments, wherein the one or more support areas are formed on the nonwoven fibrous web by a hydroentangling process.

Forty-fifth example embodiment: The multilayer nonwoven material of any one of the thirty-second through forty-third example embodiments, wherein the one or more support areas are formed on the nonwoven fibrous web by one or both of a bonding process and an embossment process.

Forty-sixth example embodiment: A garment, comprising a layer formed with the multilayer nonwoven material of any one of the thirty-second through forty-fifth example embodiments.

1. A multilayer nonwoven material, comprising:
a laminate nonwoven fibrous web defining a lateral direction and a longitudinal direction, the lateral and longitudinal directions being perpendicular, the laminate nonwoven fibrous web comprising a first web and a second web, the laminate nonwoven fibrous web comprising a perforated zone with a plurality of apertures that are distributed across a width of the perforated zone along the lateral direction and across a length of the perforated zone along the longitudinal direction,
wherein the laminate nonwoven fibrous web comprises one or more support areas, fibers of the first web entangled within discrete portions of the first web and consolidated with fibers of the second web to a greater extent at the one or more support areas than at the areas of the laminate nonwoven fibrous web adjacent to the one or more support areas, a collective area of the one or more support areas on the laminate nonwoven fibrous web being no less than a half percent and no greater than thirty percent of a total area of the laminate nonwoven fibrous web.

2. The multilayer nonwoven material of claim 1, wherein the one or more support areas differ from the areas of the laminate nonwoven fibrous web adjacent to the one or more support areas by one or more of basis weight, density, caliper, and fiber-to-fiber bonding.

3. The multilayer nonwoven material of claim 1, wherein the collective area of the one or more support areas on the laminate nonwoven fibrous web is no less than five percent and no greater than twenty percent of a total area of the laminate nonwoven fibrous web.

4. The multilayer nonwoven material of claim 1, wherein a basis weight of the laminate nonwoven fibrous web at the one or more support areas is greater than the basis weight of the laminate nonwoven fibrous web at the areas of the laminate nonwoven fibrous web adjacent to the one or more support areas.

5. The multilayer nonwoven material of claim 1, wherein a caliper of the laminate nonwoven fibrous web at the one or more support areas is greater than the caliper of the laminate nonwoven fibrous web at the areas of the laminate nonwoven fibrous web adjacent to the one or more support areas.

6. The multilayer nonwoven material of claim 1, wherein the laminate nonwoven fibrous web comprises a first surface and a second surface, the first surface being opposite the second surface on the laminate nonwoven fibrous web, the plurality of apertures extending between the first and second surfaces of the laminate nonwoven fibrous web, the one or more support areas filled by the fibers of the first web at the first surface and consolidated with the fibers of the second web between the first and second surfaces of the laminate nonwoven fibrous web.

7. The multilayer nonwoven material of claim 1, wherein the laminate nonwoven fibrous web comprises a first surface and a second surface, the first surface being opposite the second surface on the laminate nonwoven fibrous web, the laminate nonwoven fibrous web comprising a plurality of nodes extending away from a base plane on the first surface.

8. The multilayer nonwoven material of claim 7, wherein the plurality of nodes are hollow.

9. The multilayer nonwoven material of claim 7, wherein the plurality of nodes extend from the first surface by a height of the plurality of nodes, the one or more support areas extend from the first surface by a height of the one or more support areas, and an average height of the plurality of nodes is greater than an average height of the one or more support areas.

10. The multilayer nonwoven material of claim 7, the fibers of the first web are entangled within the discrete portions of the first web and consolidated with the fibers of the second web to a greater extent at the one or more support areas than at the plurality of nodes.

11. The multilayer nonwoven material of claim 1, wherein the one or more support areas comprise a plurality of support areas, and the plurality of support areas are discrete from one another on the laminate nonwoven fibrous web.

12. The multilayer nonwoven material of claim 11, wherein:
the width of the perforated zone along the lateral direction is less than a width of the nonwoven fibrous web along the lateral direction;
the plurality of support areas are elongated along the lateral direction;
the plurality of support areas comprises a first plurality of support areas and a second plurality of support areas;
the first plurality of support areas are positioned between a first side portion of the perforated zone and a first side portion of the nonwoven fibrous web along the lateral direction, the first plurality of support areas distributed along the longitudinal direction; and
the second plurality of support areas are positioned between a second side portion of the perforated zone and a second side portion of the nonwoven fibrous web along the lateral direction, the second plurality of support areas distributed along the longitudinal direction.

13. The multilayer nonwoven material of claim 12, wherein:
a first side lane extends between the first side portion of the perforated zone and the first side portion of the nonwoven fibrous web along the lateral direction;
a second side lane extends between the second side portion of the perforated zone and the second side portion of the nonwoven fibrous web along the lateral direction;
a length of each of the first plurality of support areas along the lateral direction is no less than twenty-five percent and no greater than one hundred percent of a length of the first side lane along the lateral direction; and
a length of each of the second plurality of support areas along the lateral direction is no less than twenty-five percent and no greater than one hundred percent of a length of the second side lane along the lateral direction.

14. The multilayer nonwoven material of claim 11, wherein the plurality of support areas are arranged parallel with adjacent support areas spaced apart by no less than about ten millimeters and no greater than about two hundred millimeters.

15. The multilayer nonwoven material of claim 11, wherein:
the nonwoven fibrous web comprises a first surface and a second surface, the first surface being opposite the second surface on the nonwoven fibrous web;
a distal end portion of each of the plurality of support areas is spaced from the first surface of the nonwoven fibrous web by a height of each of the plurality of support areas; and
an average height of the plurality of support areas is no less than a half millimeter and no greater than five millimeters.

16. The multilayer nonwoven material of claim 1, wherein the one or more support areas are formed on the nonwoven fibrous web by a hydroentangling process.

17. The multilayer nonwoven material of claim 1, wherein the one or more support areas are formed on the nonwoven fibrous web by one or both of a bonding process and an embossment process.

18. A garment, comprising a layer formed with the multilayer nonwoven material of claim 1.

19. A multilayer nonwoven material, comprising:
a laminate nonwoven fibrous web defining a lateral direction and a longitudinal direction, the lateral and longitudinal directions being perpendicular, the laminate nonwoven fibrous web comprising a first web and a second web, the laminate nonwoven fibrous web comprising a perforated zone with a plurality of apertures that are distributed across a width of the perforated zone along the lateral direction and across a length of the perforated zone along the longitudinal direction,
wherein the laminate nonwoven fibrous web comprises a first surface and a second surface, the first surface being opposite the second surface on the laminate nonwoven fibrous web, the laminate nonwoven fibrous web comprising a plurality of nodes extending away from a base plane on the first surface,
wherein the laminate nonwoven fibrous web comprises one or more support areas, fibers of the first web entangled within discrete areas of the first web and consolidated with fibers of the second web to a greater extent at the one or more support areas than at the areas of the laminate nonwoven fibrous web adjacent to the one or more support areas.

20. (canceled)

21. (canceled)

22. (canceled)

23. (canceled)

24. (canceled)

25. (canceled)

26. (canceled)

27. (canceled)

28. (canceled)

29. The multilayer nonwoven material of claim 19, wherein the plurality of apertures provide a percent open area for the perforated zone no less than about ten percent and no greater than about sixty percent.

30. The multilayer nonwoven material of claim 29, wherein an area of each of the plurality of apertures is no less than about eight millimeters squared and no greater than about eighteen millimeters squared.

31. (canceled)

32. A multilayer nonwoven material, comprising:
a laminate nonwoven fibrous web defining a lateral direction and a longitudinal direction, the lateral and longitudinal directions being perpendicular, the laminate nonwoven fibrous web comprising a first web and a second web, the laminate nonwoven fibrous web comprising a perforated zone with a plurality of apertures that are distributed across a width of the perforated zone along the lateral direction and across a length of the perforated zone along the longitudinal direction,
wherein the laminate nonwoven fibrous web comprises a first surface and a second surface, the first surface being opposite the second surface on the laminate nonwoven fibrous web, the laminate nonwoven fibrous web comprising a plurality of hollow nodes extending away from a base plane on the first surface,
wherein the laminate nonwoven fibrous web comprises one or more support areas, fibers of the first web entangled at the one or more support areas of the first web and consolidated with fibers of the second web to a greater extent at the one or more support areas than at the areas of the laminate nonwoven fibrous web adjacent to the one or more support areas, the one or more support areas filled by the fibers of the first web at the first surface and consolidated with the fibers of the second web between the first and second surfaces of the laminate nonwoven fibrous web.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)
